(19) United States
(12) Patent Application Publication
(10) Pub. No.: US 2022/0280572 A1
(43) Pub. Date: Sep. 8, 2022

(54) METHODS OF TREATING VASCULAR DISEASES

(71) Applicant: Astellas Institute for Regenerative Medicine, Westborough, MA (US)

(72) Inventors: Nagisa Sakurai, Westborough, MA (US); Maria Mirotsou, Westborough, MA (US); Nutan Prasain, Westborough, MA (US); Amrita Singh, Westborough, MA (US); Robert Lanza, Westborough, MA (US)

(21) Appl. No.: 17/632,708

(22) PCT Filed: Aug. 27, 2020
(86) PCT No.: PCT/US2020/048080

§ 371 Date: Feb. 3, 2022

Related U.S. Application Data
(60) Provisional application No. 62/892,712, filed on Aug. 28, 2019.

(51) Int. Cl. A61K 35/28; A61K 35/44; C12N 5/0789; C12N 5/071; A61P 9/12

(52) U.S. Cl. CPC A61K 35/28; A61K 35/44; C12N 5/0647; C12N 5/069; A61P 9/12

(57) ABSTRACT

The present invention provides methods for treating vascular diseases with hemogenic endothelial cells (HEs) obtained in vitro from pluripotent stem cells. The present invention also provides compositions and methods of producing the HEs.

### RELATED APPLICATIONS

This application claims the benefit under 35 U.S.C. § 119(e) of U.S. Provisional Application No. 62/892,712, filed Aug. 28, 2019, which is hereby incorporated by reference in its entirety.

### FIELD OF THE INVENTION

The present invention relates to methods of treating vascular diseases with hemogenic endothelial cells obtained by in vitro differentiation of pluripotent stem cells.

### BACKGROUND

Cardiovascular disease is a class of diseases that involves the heart or blood vessels and is the leading cause of death worldwide. In the United States alone, approximately 84 million people suffer from cardiovascular disease, and almost one out of every three deaths results from cardiovascular disease.

Pulmonary hypertension (PH) is a condition characterized by increased pressure in the main pulmonary artery. A deadly form of PH is pulmonary arterial hypertension (PAH) and typically leads to death within an average of 2.8 years from diagnosis. PAH is characterized by vasoconstriction and remodeling of the pulmonary vessels. Standard available therapies may improve the quality of life and prognosis of patients but typically do not directly prevent the pathogenic remodeling process and may sometimes have serious side effects.

Peripheral arterial disease (PAD) is an abnormal narrowing and obstruction of the arteries other than those of the cerebral and coronary circulations. Critical limb ischemia (CLI) is a serious form of PAD that results in severe blockage in the arteries of the lower extremities. CLI is associated with major limb loss, myocardial infarction, stroke, and death. To date, there is no effective treatment for CLI.

Coronary artery disease is the most common form of cardiovascular disease and is caused by reduced blood flow and oxygen to the heart muscle due to atherosclerosis of the arteries of the heart. Patients with coronary artery disease often receive balloon angioplasty or stents to clear occluded arteries. Some undergo coronary artery bypass surgery at high expense and risk.

Thus, there is a need for better methods for treating and preventing vascular diseases.

### SUMMARY OF THE INVENTION

The present invention provides a methods of treating a vascular disease comprising administering to a subject a composition comprising hemogenic endothelial cells (HEs) obtained by in vitro differentiation of pluripotent stem cells.

In an embodiment, the vascular disease is selected from the group consisting of coronary artery diseases (e.g., arteriosclerosis, atherosclerosis, and other diseases or injuries of the arteries, arterioles and capillaries or related complaint), myocardial infarction, (e.g. acute myocardial infarction), organizing myocardial infarct, ischemic heart disease, arrhythmia, left ventricular dilatation, emboli, heart failure, congestive heart failure, subendocardial fibrosis, left or right ventricular hypertrophy, myocarditis, chronic coronary ischemia, dilated cardiomyopathy, restenosis, arrhythmia, angina, hypertension (e.g. pulmonary hypertension, glomerular hypertension, portal hypertension), myocardial hypertrophy, peripheral arterial disease including critical limb ischemia, cerebrovascular disease, renal artery stenosis, aortic aneurysm, pulmonary heart disease, cardiac dysrhythmias, inflammatory heart disease, congenital heart disease, rheumatic heart disease, diabetic vascular diseases, and endothelial lung injury diseases (e.g., acute lung injury (ALI), acute respiratory distress syndrome (ARDS)). In a specific embodiment, the vascular disease is pulmonary hypertension. In another embodiment, the vascular disease is pulmonary arterial hypertension.

In an embodiment of any of the methods disclosed herein, the mean pulmonary (artery) pressure is reduced in the subject.

The present invention also provides a method of increasing blood flow in pulmonary arteries comprising administering to a subject a composition comprising HEs obtained by in vitro differentiation of pluripotent stem cells. In an embodiment, the subject has pulmonary hypertension. In a specific embodiment, the subject has pulmonary arterial hypertension.

The present invention further provides a method of reducing blood pressure in a subject comprising administering to the subject a composition comprising HEs obtained by in vitro differentiation of pluripotent stem cells. In an embodiment, the subject has pulmonary hypertension. In a specific embodiment, the subject has pulmonary arterial hypertension. In another embodiment, the blood pressure is diastolic pressure. In yet another embodiment, the blood pressure is systolic pressure. In a further embodiment, the blood pressure is mean pulmonary (artery) pressure. Moreover, the blood pressure may be reduced by at least 20% in the subject by any of the methods of the present invention.

In an embodiment, the pluripotent stem cells disclosed herein are embryonic stem cells. In another embodiment, the pluripotent stem cells disclosed herein are induced pluripotent stem cells.

In yet another embodiment, the HEs disclosed herein are obtained by culturing the pluripotent stem cells under adherent conditions in a differentiation medium in the absence of methylcellulose. In another embodiment, the HEs disclosed herein are obtained by in vitro differentiation of pluripotent stem cells without embryoid body formation.

In any of the methods provided herein, the subject may be a human. Additionally, the pluripotent stem cells disclosed herein may be human pluripotent stem cells. Furthermore, the HEs disclosed herein may be human HEs.

The HEs disclosed herein may be positive for at least one microRNA (miRNA) selected from the group consisting of miRNA-126, mi-RNA-24, miRNA-196-b, miRNA-214, miRNA-199a-3p, miRNA-335, hsa-miR-11399, hsa-miR-196b-3p, hsa-miR-5690, and hsa-miR-7151-3p. In an embodiment, the HEs are positive for (i) miRNA-214, miRNA-199a-3p, and miRNA-335 and/or (ii) hsa-miR-11399, hsa-miR-196b-3p, hsa-miR-5690, and hsa-miR-7151-3p. In another embodiment, the HEs are positive for (i) miRNA-126, miRNA-24, miRNA-196-b, miRNA-214, miRNA-199a-3p, and miRNA-335 and/or (ii) hsa-miR-11399, hsa-miR-196b-3p, hsa-miR-5690, and hsa-miR-7151-3p. In an embodiment, the HEs are positive for miRNA-214.

In any of the embodiments, the HEs disclosed herein may be negative for at least one miRNA selected from the group consisting of miRNA-367, miRNA-302a, miRNA-302b, miRNA-302c, miRNA-223, and miRNA-142-3p. In an embodiment, the HEs are negative for miRNA-223, and miRNA-142-3p. In another embodiment, the HEs are negative for miRNA-367, miRNA-302a, miRNA-302b, miRNA-302c, miRNA-223, and miRNA-142-3p.

In an embodiment, the HEs are positive for miRNA-214, miRNA-199a-3p, and miRNA-335, and negative for miRNA-223, and miRNA-142-3p.

In an embodiment, any of the HEs disclosed herein express at least one cell surface marker selected from the group consisting of CD31/PECAM1, CD309/KDR, CD144, CD34, CXCR4, CD146, Tie2, CD140b, CD90, CD271, and CD105. In an embodiment, the HEs of the invention express CD146, CXCR4, CD309/KDR, CD90, and CD271. In another embodiment, the HEs of the invention express CD146. In an embodiment, the HEs of the invention express CD31/PECAM1, CD309/KDR, CD144, CD34, and CD105.

In an embodiment, the HEs exhibit limited or no detection of at least one cell surface marker selected from the group consisting of CD34, CXCR7, CD43 and CD45. In another embodiment, the HEs exhibit limited or no detection of CXCR7, CD43, and CD45. In another embodiment, the HEs exhibit limited or no detection of CD43 and CD45.

In an embodiment, the HEs of the invention are CD43(−), CD45(−), and/or CD146 (+). In another embodiment, HEs express CD31, Calponin (CNN1), and NG2, and therefore have the potential to differentiate into endothelial (CD31+), smooth muscle (Calponin+) and/or pericyte (NG2+) cells.

In an embodiment, CD144 (VECAD)-expressing HEs are isolated from the HEs of the inventions. In an embodiment, the isolated CD144 (VECAD)-expressing HE cells further express CD31 and/or CD309/KDR (FLK-1). In another embodiment, the isolated CD144 (VECAD)-expressing HE cells further express at least one gene listed in Table 22 and Table 23. In another embodiment, the isolated CD144 (VECAD)-expressing HE cells further express at least one cell marker selected from the group consisting of PLVAP, GJA4, ESAM, EGFL7, KDR/VEGFR2, and ESAM. In an embodiment, the isolated CD144 (VECAD)-expressing HE cells further express at least one cell marker selected from the group consisting of SOX9, PDGFRA, and EGFRA. In another embodiment, the isolated CD144 (VECAD)-expressing HE cells further express at least one cell marker selected from the group consisting of KDR/VEGFR2, NOTCH4, collagen I, and collagen IV. In an embodiment, the composition comprising CD144 (VECAD)-expressing HEs isolated from the HEs of the invention substantially lack CD144 (VECAD)-negative HEs.

Accordingly, the present invention also provides a composition comprising HEs obtained by in vitro differentiation of pluripotent stem cells disclosed herein. The present invention further provides a pharmaceutical composition comprising HEs obtained by in vitro differentiation of pluripotent stem cells disclosed herein and a pharmaceutically acceptable carrier.

### BRIEF DESCRIPTION OF THE DRAWINGS

FIG. 1 is an overview of an exemplary method for producing HEs.

FIG. 2 is an overview of an exemplary method for producing hemangioblasts (HBs).

FIG. 3 are bar graphs of PDGFRa, HAND1, FOXF1, APLNR, PECAM/CD31 expression in cells over the course the differentiation process from ES cells (day −1). Time points tested were at day −1 (ES cells), day 2 (D2), day 4 (D4), and day 6 (D6).

FIG. 4A shows a graph of CD31, CD43, CD34, KDR, CXCR4, CD144, CD146, and CD105 expression in J1-HE cells (red, left bar) and GMP1-HE cells (blue, right bar) obtained at day 6 of the differentiation process.

FIG. 4B shows graphs of CD31, VECAD, CD34, FLK1 (KDR), CD105, CD146, CD43, CXCR4, CD140b (PDGFRb), and NG2 in J1-HE cells and GMP1-HE cells obtained at day 6 of the differentiation process. Red is stained, gray is unstained control.

FIG. 5 shows graphs of J1-HE and GMP1-HE populations gated for CD31 positive (red) and negative (blue) cells and their respective expression of FLK1/CD309, CD144/VECAD, CD34, CD105, and CD43.

FIG. 6 shows representative images of GMP-1-derived HEs stained with CD31, NG2, or CNN1 antibodies (bottom panels). HUVEC cells were used for comparison (top panels).

FIG. 7 is a TSNE plot of miRNAs from HUVEC cells, J1 hESCs, J1-HEs, or J1-HBs.

FIG. 8 shows the effect of HB (VPC1) and HE (VPC2) on the rate of survival of sugen-hypoxia induced PAH rat.

FIG. 9A shows 9 clusters by unsupervised clustering of HUVEC, iPSC (GMP1), and GMP1-HEs.

FIG. 9B shows the percentage of HUVEC, iPSC (GMP1) and GMP1-HE (“VPC-feeder Active”) in each of the 9 clusters.

FIG. 9C shows distinct clustering of HUVEC, iPSC (GMP1) and GMP1-HE (“VPC-feeder Active”).

FIG. 10 shows three clusters identified by the expression of VECAD/CDH5.

FIG. 11A shows the right ventricle systolic pressure (RVSP) in MCT rats treated with vehicle (control medium), sildenafil (positive control), J1-HE (2.5×10⁶ cells), and GMP1-HE (2.5×10⁶ cells), as well as in the non-MCT treated control (Cont(Nx)).

FIG. 11B shows the Fulton's Index (RV/LV+S) in MCT rats treated with vehicle (control medium), sildenafil (positive control), J1-HE (2.5×10⁶ cells), and GMP1-HE (2.5×10⁶ cells), as well as in the non-MCT treated control (Cont(Nx)).

FIG. 11C shows the pulmonary vascular resistance index (PVR Index) in MCT rats treated with vehicle (control medium), sildenafil (positive control), J1-HE (2.5×10⁶ cells), and GMP1-HE (2.5×10⁶ cells), as well as in the non-MCT treated control (Cont(Nx)).

FIG. 11D shows the number of thickened small vessels in MCT rats treated with vehicle (control medium), sildenafil (positive control), J1-HE (2.5×10⁶ cells), and GMP1-HE (2.5×10⁶ cells), as well as in the non-MCT treated control (Cont(Nx)).

FIG. 12A shows the mean pulmonary arterial pressure (mPAP) in Sugen-treated rats treated with vehicle (negative control), J1-HE (2.5 million cells), and GMP1-HE (2.5 million cells), as well as in the non-Sugen treated control (Nx).

FIG. 12B shows the right ventricle systolic pressure (RVSP) in Sugen-treated rats treated with vehicle (negative control), J1-HE (2.5 million cells), and GMP1-HE (2.5 million cells), as well as in the non-Sugen treated control (Nx).

FIG. 12C shows the Fulton's index (RV/LV+S) in Sugen-treated rats treated with vehicle (negative control), J1-HE (2.5 million cells), and GMP1-HE (2.5 million cells), as well as in the non-Sugen treated control (Nx).

FIG. 12D shows the cardiac output in Sugen-treated rats treated with vehicle (negative control), J1-HE (2.5 million cells), and GMP1-HE (2.5 million cells), as well as in the non-Sugen treated control (Nx).

FIG. 13A shows the mean pulmonary arterial pressure (mPAP) in Sugen-treated rats treated with vehicle (negative control), GMP1-HE (1 million cells), GMP1-HE (2.5 million cells), GMP1-HE (5 million cells), and sildenafil (positive control), as well as in the non-Sugen treated control (Nx).

FIG. 13B shows the right ventricle systolic pressure (RVSP) in Sugen-treated rats treated with vehicle (negative control), GMP1-HE (1 million cells), GMP1-HE (2.5 million cells), GMP1-HE (5 million cells), and sildenafil (positive control), as well as in the non-Sugen treated control (Nx).

FIG. 13C shows the Fulton's index (RV/LV+S) in Sugen-treated rats treated with vehicle (negative control), GMP1-HE (1 million cells), GMP1-HE (2.5 million cells), GMP1-HE (5 million cells), and sildenafil (positive control), as well as in the non-Sugen treated control (Nx).

FIG. 13D shows the cardiac output in Sugen-treated rats treated with vehicle (negative control), GMP1-HE (1 million cells), GMP1-HE (2.5 million cells), GMP1-HE (5 million cells), and sildenafil (positive control), as well as in the non-Sugen treated control (Nx).

FIG. 14A shows histological images of lung tissue in Sugen-treated rats treated with vehicle (negative control), GMP1-HE (1 million cells), GMP1-HE (2.5 million cells), and GMP1-HE (5 million cells), as well as in the non-Sugen treated control (Nx).

FIG. 14B shows the lung vessel wall thickness in Sugen-treated rats treated with vehicle (negative control), GMP1-HE (1 million cells), GMP1-HE (2.5 million cells), GMP1-HE (5 million cells), and sildenafil (positive control), as well as in the non-Sugen treated control (Nx).

FIG. 14C shows the percentage of muscular, semi-muscular, and non-muscular lung vessels in Sugen-treated rats treated with vehicle (negative control), GMP1-HE (1 million cells), GMP1-HE (2.5 million cells), GMP1-HE (5 million cells), and sildenafil (positive control), as well as in the non-Sugen treated control (Nx).

FIG. 15A shows histological images of lung tissue in Sugen-treated rats treated with vehicle (negative control), J1-HE (2.5 million cells), and GMP1-HE (2.5 million cells), as well as in the non-Sugen treated control (Nx).

FIG. 15B shows lung vessel wall thickness in Sugen-treated rats treated with vehicle (negative control), J1-HE (2.5 million cells), and GMP1-HE (2.5 million cells), as well as in the non-Sugen treated control (Nx).

FIG. 15C shows the percentage of muscular, semi-muscular, and non-muscular lung vessels in Sugen-treated rats treated with vehicle (negative control), J1-HE (2.5 million cells), and GMP1-HE (2.5 million cells), as well as in the non-Sugen treated control (Nx).

FIG. 16A shows a microCT-scanned image of a normal lung in a non-Sugen-treated rat (Nx control).

FIG. 16B shows a microCT-scanned image of a lung in a SuHx rat treated with a vehicle (negative control).

FIG. 16C shows a microCT-scanned image of a lung in a SuHx rat treated with 1 million GMP-1 HE cells.

FIG. 16D shows a microCT-scanned image of a lung in a SuHx rat treated with 5 million GMP-1 HE cells.

FIG. 16E shows a microCT-scanned image of a lung in a SuHx rat treated with sildenafil.

FIG. 17 shows the expression of CD31 and VECAD in unsorted HE cells (“unsorted”) and in VECAD negative (− Fraction) and VECAD positive (+ Fraction) cells after sorting for VECAD expression.

FIG. 18A shows the mean pulmonary arterial pressure (mPAP) in Sugen-treated rats treated with vehicle (negative control), unsorted GMP1-HE, and sorted VECAD+ GMP1-HE, as well as in the non-Sugen treated control (Nx).

FIG. 18B shows the right ventricle systolic pressure (RVSP) in Sugen-treated rats treated with vehicle (negative control), unsorted GMP1-HE, and sorted VECAD+ GMP1-HE, as well as in the non-Sugen treated control (Nx).

FIG. 18C shows the Fulton's index (RV/LV+S) in Sugen-treated rats treated with vehicle (negative control), unsorted GMP1-HE, and sorted VECAD+ GMP1-HE, as well as in the non-Sugen treated control (Nx).

FIG. 18D shows the cardiac output in Sugen-treated rats treated with vehicle (negative control), unsorted GMP1-HE, and sorted VECAD+ GMP1-HE, as well as in the non-Sugen treated control (Nx).

FIG. 18E shows histological images of lung tissue in Sugen-treated rats treated with vehicle (negative control), unsorted GMP1-HE, and sorted VECAD+ GMP1-HE, as well as in the non-Sugen treated control (Nx).

FIG. 18F shows the lung vessel wall thickness in Sugen-treated rats treated with vehicle (negative control), unsorted GMP1-HE, and sorted VECAD+ GMP1-HE, as well as in the non-Sugen treated control (Nx).

FIG. 18G shows the percentage of muscular, semi-muscular, and non-muscular lung vessels in in Sugen-treated rats treated with vehicle (negative control), unsorted GMP1-HE, and sorted VECAD+ GMP1-HE, as well as in the non-Sugen treated control (Nx).

FIG. 19 shows FLK1/KDR expression of CD31+/VECAD+ populations in J1-HEs, GMP1-HEs, and HUVEC cells.

### DETAILED DESCRIPTION OF THE INVENTION

In order that the present invention may be more readily understood, certain terms are first defined. It should also be noted that whenever a value or range of values of a parameter are recited, it is intended that values and ranges intermediate to the recited values are also part of this invention.

In the following description, for purposes of explanation, specific numbers, materials, and configurations are set forth in order to provide a thorough understanding of the invention. It will be apparent, however, to one having ordinary skill in the art that the invention may be practiced without these specific details. In some instances, well-known features may be omitted or simplified so as not to obscure the present invention. Furthermore, reference in the specification to phrases such as “one embodiment” or “an embodiment” mean that a particular feature, structure, or characteristic described in connection with the embodiment is included in at least one embodiment of the invention. The appearances of phrases such as “in one embodiment” in various places in the specification are not necessarily all referring to the same embodiment.

The articles “a” and “an” are used herein to refer to one or to more than one (i.e., to at least one) of the grammatical object of the article. By way of example, “an element” refers to one element or more than one element.

The term “comprising” or “comprises” is used herein in reference to compositions, methods, and respective component(s) thereof, that are essential to the disclosure, yet open to the inclusion of unspecified elements, whether essential or not.

“Pluripotent stem cell,” as used herein, refers broadly to a cell capable of prolonged or virtually indefinite proliferation in vitro while retaining their undifferentiated state, exhibiting normal karyotype (e.g., chromosomes), and having the capacity to differentiate into all three germ layers (i.e., ectoderm, mesoderm and endoderm) under the appropriate conditions. Pluripotent stem cells are typically defined functionally as stem cells that are: (a) capable of inducing teratomas when transplanted in immunodeficient (SCID) mice; (b) capable of differentiating to cell types of all three germ layers (e.g., can differentiate to ectodermal, mesodermal, and endodermal cell types); and (c) express one or more markers of embryonic stem cells (e.g., Oct 4, alkaline phosphatase. SSEA-3 surface antigen, SSEA-4 surface antigen, nanog, TRA-1-60, TRA-1-81, SOX2, REX1, etc.). In certain embodiments, pluripotent stem cells express one or more markers selected from the group consisting of: OCT-4, alkaline phosphatase, SSEA-3, SSEA-4, TRA-1-60, and TRA-1-81. Exemplary pluripotent stem cells can be generated using, for example, methods known in the art.

Pluripotent stem cells include, but are not limited to, embryonic stem cells, induced pluripotent stem (iPS) cells, embryo-derived cells (EDCs), adult stem cells, hematopoietic cells, fetal stem cells, mesenchymal stem cells, postpartum stem cells, or embryonic germ cells. In an embodiment, the pluripotent stem cells are mammalian pluripotent stem cells. In another embodiment, the pluripotent stem cells are human pluripotent stem cells including, but not limited to, human embryonic stem (hES) cells, human induced pluripotent stem (iPS) cells, human adult stem cells, human hematopoietic stem cells, human fetal stem cells, human postpartum stem cells, human multipotent stem cells, or human embryonic germ cells. In one embodiment, the pluripotent stem cell is human embryonic stem cell. In another embodiment, the pluripotent stem cell is human induced pluripotent stem cell. In another embodiment, the pluripotent stem cells may be a pluripotent stem cell listed in the Human Pluripotent Stem Cell Registry, hPSCreg. Pluripotent stem cells may be genetically modified or otherwise modified to increase longevity, potency, homing, to prevent or reduce alloimmune responses or to deliver a desired factor in cells that are differentiated from such pluripotent cells.

The pluripotent stem cells may be from any species. Embryonic stem cells have been successfully derived from, for example, mice, multiple species of non-human primates, and humans, and embryonic stem-like cells have been generated from numerous additional species. Thus, one of skill in the art can generate embryonic stem cells and embryo-derived stem cells from any species, including but not limited to, human, non-human primates, rodents (mice, rats), ungulates (cows, sheep, etc.), dogs (domestic and wild dogs), cats (domestic and wild cats such as lions, tigers, cheetahs), rabbits, hamsters, gerbils, squirrel, guinea pig, goats, elephants, panda (including giant panda), pigs, raccoon, horse, zebra, marine mammals (dolphin, whales, etc.) and the like.

“Embryo” or “embryonic,” as used herein refers broadly to a developing cell mass that has not implanted into the uterine membrane of a maternal host. An “embryonic cell” is a cell isolated from or contained in an embryo. This also includes blastomeres, obtained as early as the two-cell stage, and aggregated blastomeres.

“Embryonic stem cells” (ES cells), as used herein, refers broadly to cells derived from the inner cell mass of blastocysts or morulae that have been serially passaged as cell lines. The ES cells may be derived from fertilization of an egg cell with sperm or DNA, nuclear transfer, parthenogenesis, or by means to generate ES cells with homozygosity in the HLA region. ES cells may also refer to cells derived from a zygote, blastomeres, or blastocyst-staged mammalian embryo produced by the fusion of a sperm and egg cell, nuclear transfer, parthenogenesis, or the reprogramming of chromatin and subsequent incorporation of the reprogrammed chromatin into a plasma membrane to produce a cell, optionally without destroying the remainder of the embryo. Embryonic stem cells, regardless of their source or the particular method used to produce them, may be identified based on one or more of the following features: (i) ability to differentiate into cells of all three germ layers, (ii) expression of at least Oct-4 and alkaline phosphatase, and (iii) ability to produce teratomas when transplanted into immunocompromised animals.

“Embryo-derived cells” (EDC), as used herein, refers broadly to morula-derived cells, blastocyst-derived cells including those of the inner cell mass, embryonic shield, or epiblast, or other pluripotent stem cells of the early embryo, including primitive endoderm, ectoderm, and mesoderm and their derivatives. “EDC” also including blastomeres and cell masses from aggregated single blastomeres or embryos from varying stages of development, but excludes human embryonic stem cells that have been passaged as cell lines.

“Induced pluripotent stem cells” or “iPS cells,” as used herein, generally refer to pluripotent stem cells obtained by reprogramming a somatic cell. An iPS cell may be generated by expressing or inducing expression of a combination of factors (“reprogramming factors”), for example, Oct 4 (sometimes referred to as Oct 3/4), Sox2, Myc (eg. c-Myc or any Myc variant), Nanog, Lin28, and Klf4, in a somatic cell. In an embodiment, the reprogramming factors comprise Oct 4, Sox2, c-Myc, and Klf4. In another embodiment, reprogramming factors comprise Oct 4, Sox2, Nanog, and Lin28. In certain embodiments, at least two reprogramming factors are expressed in a somatic cell to successfully reprogram the somatic cell. In other embodiments, at least three reprogramming factors are expressed in a somatic cell to successfully reprogram the somatic cell. In other embodiments, at least four reprogramming factors are expressed in a somatic cell to successfully reprogram the somatic cell. In another embodiment, at least five reprogramming factors are expressed in a somatic cell to successfully reprogram the somatic cell. In yet another embodiment, at least six reprogramming factors are expressed in the somatic cell, for example, Oct 4, Sox2, c-Myc, Nanog, Lin28, and Klf4. In other embodiments, additional reprogramming factors are identified and used alone or in combination with one or more known reprogramming factors to reprogram a somatic cell to a pluripotent stem cell.

iPS cells may be generated using fetal, postnatal, newborn, juvenile, or adult somatic cells. Somatic cells may include, but are not limited to, fibroblasts, keratinocytes, adipocytes, muscle cells, organ and tissue cells, and various blood cells including, but not limited to, hematopoietic cells (eg. hematopoietic stem cells). In an embodiment, the somatic cells are fibroblast cells, such as a dermal fibroblast, synovial fibroblast, or lung fibroblast, or a non-fibroblastic somatic cell.

iPS cells may be obtained from a cell bank. Alternatively, IPS cells may be newly generated by methods known in the art. iPS cells may be specifically generated using material, from a particular patient or matched donor with the goal of generating tissue-matched cells. In an embodiment, iPS cells may be universal donor cells that are not substantially immunogenic.

The induced pluripotent stem cell may be produced by expressing or inducing the expression of one or more reprogramming factors in a somatic cell. Reprogramming factors may be expressed in the somatic cell by infection using a viral vector, such as a retroviral vector or a lentiviral vector. CRISPR/Talen/zinc-finger nucleases (XFNs) may also be used. Also, reprogramming factors may be expressed in the somatic cell using a non-integrative vector, such as an episomal plasmid, or RNA. When reprogramming factors are expressed using non-integrative vectors, the factors may be expressed in the cells using electroporation, transfection, or transformation of the somatic cells with the vectors. For example, in mouse cells, expression of four factors (Oct3/4, Sox2, c-myc, and Klf4) using integrative viral vectors is sufficient to reprogram a somatic cell. In human cells, expression of four factors (Oct3/4, Sox2, NANOG, and Lin28) using integrative viral vectors is sufficient to reprogram a somatic cell.

Expression of the reprogramming factors may be induced by contacting the somatic cells with at least one agent, such as a small organic molecule agents, that induce expression of reprogramming factors.

The somatic cell may also be reprogrammed using a combinatorial approach wherein the reprogramming factor is expressed (e.g., using a viral vector, plasmid, and the like) and the expression of the reprogramming factor is induced (e.g. using a small organic molecule.)

Once the reprogramming factors are expressed or induced in the cells, the cells may be cultured. Over time, cells with ES characteristics appear in the culture dish. The cells may be chosen and subcultured based on, for example, ES cell morphology, or based on expression of a selectable or detectable marker. The cells may be cultured to produce a culture of cells that resemble ES cells.

To confirm the pluripotency of the iPS cells, the cells may be tested in one or more assays of pluripotency. For examples, the cells may be tested for expression of ES cell markers; the cells may be evaluated for ability to produce teratomas when transplanted into SCID mice; the cells may be evaluated for ability to differentiate to produce cell types of all three germ layers.

iPS cells may be from any species. These iPS cells have been successfully generated using mouse and human cells. Furthermore, iPS cells have been successfully generated using embryonic, fetal, newborn, and adult tissue. Accordingly, one may readily generate iPS cells using a donor cell from any species. Thus, one may generate iPS cells from any species, including but not limited to, human, non-human primates, rodents (mice, rats), ungulates (cows, sheep, etc.), dogs (domestic and wild dogs), cats (domestic and wild cats such as lions, tigers, cheetahs), rabbits, hamsters, goats, elephants, panda (including giant panda), pigs, raccoon, horse, zebra, marine mammals (dolphin, whales, etc.) and the like.

When a cell is characterized as being “positive” or “+” for a given marker, it may be a low (lo), intermediate (int), and/or high (hi) expresser of that marker depending on the degree to which the marker is present on a cell surface of a cell or within a population of cells, where the terms relate to intensity of fluorescence or other color used in the color sorting process of the cells. The distinction of lo, int, and hi will be understood in the context of the marker used on a particular cell population being sorted. When a cell is characterized as being “negative” or “−” for a given marker, it means that a cell or a population of cells may not express that marker or that the marker may be expressed at a relatively very low level by that cell or a population of cells, and that it generates a very low signal when detectably labeled.

In an embodiment of the invention, if the level of expression of a marker is greater than 60%, 70%, 80%, or 90% relative to a control, the cell or population of cells is characterized as expressing high (hi) levels of the marker. In another embodiment, if the level of expression of a marker is between about 20%, 30%, 40%, 50% to about 60% relative to a control, the cell or a population of cells is characterized as expression intermediate (int) levels of the marker. In yet another embodiment, if the level of expression of a marker is between about 2%, 5%, 10%, or 15% to about 20% relative to a control, the cell or a population of cells is characterized as expression low (10) levels of the marker. In a further embodiment, if the level of expression of a marker is less than about 2%, 1.5%, 1%, or 0.5% relative to a control, the cell or population of cells is characterized as being negative for the marker. In another embodiment, if the level of expression of a marker is lo or is less than about 2%, 1.5%, 1%, or 0.5% relative to a control, the cell or population of cells is characterized as being negative for the marker. A “control” may be any control or standard familiar to one of ordinary skill in the art useful for comparison purposes and may include a negative control or a positive control.

“Treatment” or “treating” as used herein, refers to curing, healing, alleviating, relieving, altering, remedying, ameliorating, improving, affecting, preventing, or delaying the onset of a disease or disorder, or symptoms of the disease or disorder. In the context of vascular repair, the term “treatment” or “treating” includes repairing, replacing, augmenting, improving, rescuing, repopulating, or regenerating vascular tissue.

“Hemangioblast” or “HB” as used herein, refers to a cell obtained by in vitro differentiation of pluripotent stem cells that is capable of differentiating into at least hematopoietic cells and endothelial cells. In an embodiment, hemangioblasts may be generated in vitro from pluripotent stem cells according to the methods described in, for example, U.S. Pat. Nos. 9,938,500; 9,410,123; and WO 2013/082543, all of which are incorporated herein by reference in their entirety. Further, hemangioblasts may be generated in vitro from pluripotent stem cells according to the method described in Example 2 below. In a specific embodiment, hemangioblasts are generated in vitro from pluripotent stem cells by first obtaining embryoid bodies from the pluripotent stem cells under low adherent or non-adherent conditions and culturing the embryoid bodies in a culture system comprising methylcellulose to create a three dimensional environment for the cells to form blast cells. In an embodiment, the hemangioblasts may be generated from pluripotent stem cells under normoxic conditions (eg. 5% CO₂ and 20% O₂). Hemangioblasts may also be characterized based on other structural and functional properties including, but not limited to, the expression of or lack of expression of certain DNA, RNA, microRNA or protein.

In an embodiment, the hemangioblasts are positive for at least one, at least two, at least three, at least four, or at least five cell surface markers selected from the group consisting of CD31/PECAM1, CD144/VE-cadh, CD34, CD43, and CD45. In an embodiment, the HBs are positive for CD31, CD43 and CD45. In another embodiment, the HBs are positive for CD43 and CD45. In a further embodiment, the HBs express low levels or are negative for at least one, at least 2, at least 3, at least 4, at least 5, at least 6, at least 7, or at least 8 cell surface markers selected from the group consisting of CD309/KDR, CXCR4, CXCR7, CD146, Tie2, CD140b, CD90, and CD271. In another embodiment, the HBs express low levels or are negative for CD146. In another embodiment, the HBs express low levels or are negative for Tie2, CD140b, CD90, and CD271. In yet another embodiment, the HBs express low levels or are negative for CD146, Tie2, CD140b, CD90, and CD271. In an embodiment, the HBs are positive for CD43 and CD45 and express low levels or are negative for CD146, Tie2, CD140b, CD90, and CD271.

In another embodiment, the hemangioblasts are positive for at least one, at least two, at least three, or at least 4 miRNAs selected from the group consisting of miRNA-126, miRNA-24, miRNA-223, and miRNA-142-3p. In an embodiment, the hemangioblasts are positive for miRNA-126, miRNA-24, miRNA-223, and miRNA-142-3p. In a further embodiment, the hemangioblasts are negative for at least one, at least 2, at least 3, at least 4, at least 5, at least 6, at least 7, or at least 8 miRNAs selected from the group consisting of miRNA-367, miRNA-302a, miRNA-302b, miRNA-302c, miRNA-196-b, miRNA-214, miRNA-199a-3p, and mi-RNA-335. In an embodiment, the hemangioblasts are negative for miRNA-367, miRNA-302a, miRNA-302b, miRNA-302c, miRNA-196-b, miRNA-214, miRNA-199a-3p, and mi-RNA-335. In a further embodiment, the hemangioblasts are positive for miRNA-126, miRNA-24, miRNA-223, and miRNA-142-3p and are negative for miRNA-367, miRNA-302a, miRNA-302b, miRNA-302c, miRNA-196-b, miRNA-214, miRNA-199a-3p, and mi-RNA-335.

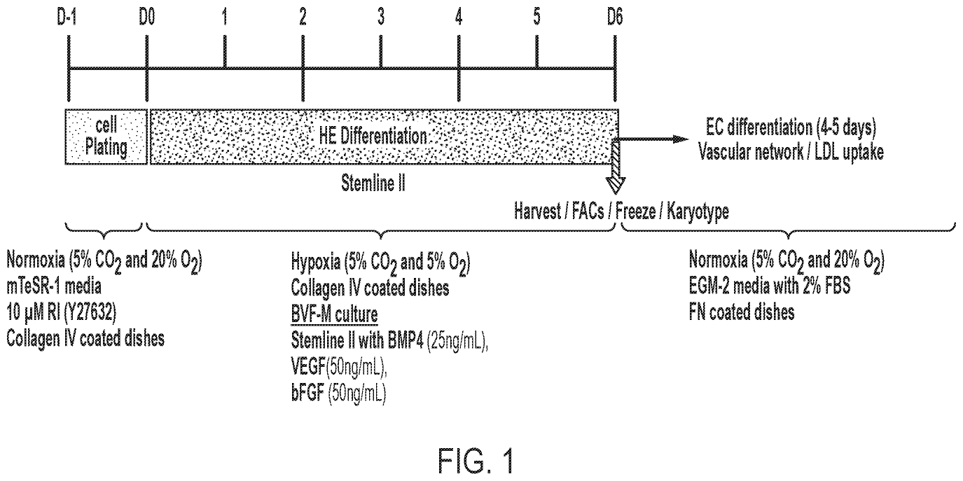

“Hemogenic endothelial cells” or “HEs”, as used herein, refers to cells obtained by in vitro differentiation of pluripotent stem cells and that have the capacity to differentiate into endothelial, smooth muscle, pericytes, hematopoietic cell and mesenchymal cell lineages. HEs may be useful for treating a vascular disease as defined herein. In an embodiment, HEs may be generated in vitro from pluripotent stem cells according to the methods described in WO 2014/100779 and U.S. Pat. No. 9,993,503, both of which are incorporated herein by reference in their entirety. In another embodiment, the HEs may be generated in vitro from pluripotent stem cells according to the methods described in Example 1 below and shown in FIG. 1.

In a specific embodiment, HEs may be generated in vitro from pluripotent stem cells without embryoid body formation or without the use of a culture system comprising methylcellulose. In an embodiment, the pluripotent stem cell is an iPS or ES cell. The pluripotent stem cell may be cultured on a feeder cell layer, preferably a human feeder cell layer, or feeder-free, for example, on an extracellular matrix such as Matrigel®. The pluripotent stem cells may be cultured under normoxic conditions (eg. 5% CO₂ and 20% O₂). For differentiation into HEs, the pluripotent stem cells may be cultured in a differentiation medium under hypoxic conditions (eg. 5% CO₂ and 5% O₂) and under adherent conditions. Adherent conditions may include culturing the cells on an extracellular matrix, such as Matrigel®, fibronectin, gelatin, and collagen IV. The differentiation medium may comprise a basal medium, such as Stemline® II Hematopoietic Stem Cell Expansion Medium (Sigma), Iscove's Modified Dulbecco's Medium (IMDM), Dulbecco's Modified Eagle's Medium (DMEM), or any other known basal medium. The differentiation medium may further comprise factors for inducing the differentiation of the pluripotent stem cells into HEs, such as bone morphogenic protein 4 (BMP4), vascular endothelial growth factor (VEGF), and fibroblast growth factor (FGF). The pluripotent stem cells may be cultured in the differentiation medium for about 1-12 days, or about 2-10 days, or about 3-8 days, or about 4, 5, 6, 7, or 8 days, or until the pluripotent stem cells differentiate into HEs. In a specific embodiment, the pluripotent stem cells are cultured in a differentiation medium for about 6 days or longer.

In an embodiment, HEs may be characterized based on certain structural and functional properties including, but not limited to, the expression of or lack of expression of certain DNA, RNA, microRNA, or protein. In an embodiment, any of the HEs disclosed herein express at least one, at least 2, at least 3, at least 4, at least 5, at least 6, at least 7, at least 8, at least 9, at least 10, or at least 11 cell surface markers selected from the group consisting of CD31/PECAM1, CD309/KDR, CD144, CD34, CXCR4, CD146, Tie2, CD140b, CD90, CD271, and CD105. In an embodiment, the HEs of the invention express CD146, CXCR4, CD309/KDR, CD90, and CD271. In another embodiment, the HEs of the invention express CD146. In another embodiment, the HEs express CD31/PECAM1, CD309/KDR, CD144, CD34, and CD105.

In an embodiment, the HEs exhibit limited or no detection of at least one, at least two, at least three, or at least four cell surface markers selected from the group consisting of CD34, CXCR7, CD43 and CD45. In another embodiment, the HEs exhibit limited or no detection of CXCR7, CD43, and CD45. In another embodiment, the HEs exhibit limited or no detection of CD43 and CD45.

In an embodiment, the HEs of the invention are CD43(−), CD45(−), and/or CD146 (+). In another embodiment HEs express CD31, Calponin (CNN1), and NG2 and therefore have the potential of differentiating further to endothelial (CD31+), smooth muscle (Calponin+) and/or pericyte (NG2+) cells.

In an embodiment, CD144 (VECAD)-expressing HEs are isolated from the HEs of the inventions. In an embodiment, the isolated CD144 (VECAD)-expressing HE cells further express CD31 and/or CD309/KDR (FLK-1). In another embodiment, the isolated CD144 (VECAD)-expressing HE cells further express at least one, at least two, at least 3, at least 4, at least 5, at least 6, at least 7, at least 8, at least 9, at least 10, at least 11, or at least 12 cell markers selected from a cell marker listed in Table 22 or Table 23. In an embodiment of the invention, the isolated CD144 (VECAD)-expressing HE cells express at least 1, at least 2, at least 3, at least 4, or at least 5 cell markers selected from the group consisting of PLVAP, GJA4, ESAM, EGFL7, KDR/VEGFR2, and ESAM. In an embodiment, the isolated CD144 (VECAD)-expressing HE cells further express at least one, at least two, or at least three cell markers selected from the group consisting of SOX9, PDGFRA, and EGFRA. In another embodiment, the isolated CD144 (VECAD)-expressing HE cells further express at least one, at least two, at least three, or at least four cell markers selected from the group consisting of KDR/VEGFR2, NOTCH4, collagen I, and collagen IV. In an embodiment, the composition comprising CD144 (VECAD)-expressing HEs isolated from the HEs of the invention substantially lack CD144 (VECAD)-negative HEs. In an embodiment, the composition comprising CD144 (VECAD)-expressing HEs comprises at least 99%, 98%, 97%, 96%, 95%, 90%, 85%, 80%, 75%, 70%, 65%, 60%, 55%, 50%, 45%, 40%, 35%, 30%, 25%, or 20% of CD144 (VECAD)-expressing HEs. In an embodiment, the composition comprising CD144 (VECAD)-expressing HEs comprises less than 1%, 2%, 3%, 4%, 5%, 10%, 15%, 20%, 25%, 30%, 35%, 40%, 45%, 50%, 55%, 60%, 65%, 70%, 75%, or 80% of CD144 (VECAD)-negative HEs.

In another embodiment, the HEs of the invention are positive for at least one, at least 2, at least 3, at least 4, at least 5, at least 6, at least 7, at least 8, at least 9, or at least 10 microRNAs (miRNAs) selected from the group consisting of miRNA-126, mi-RNA-24, miRNA-196-b, miRNA-214, miRNA-199a-3p, miRNA-335 (miRNA-335-5p and/or miRNA-335-3p), hsa-miR-11399, hsa-miR-196b-3p, hsa-miR-5690, and hsa-miR-7151-3p. In an embodiment, the HEs are positive for miRNA-214, miRNA-199a-3p, and miRNA-335 (miRNA-335-5p and/or miRNA-335-3p). In another embodiment, the HEs are positive for miRNA-126, mi-RNA-24, miRNA-196-b, miRNA-214, miRNA-199a-3p, and miRNA-335 (miRNA-335-5p and/or miRNA-335-3p). In an embodiment, the HEs are positive for miRNA-214. In another embodiment, the HEs are positive for hsa-miR-11399, hsa-miR-196b-3p, hsa-miR-5690, and hsa-miR-7151-3p. hsa-miR-11399, hsa-miR-196b-3p, hsa-miR-5690, and hsa-miR-7151-3p were identified as being uniquely expressed in populations of HEs when compared with J1 and meso 3D VPC2 cells described in U.S. Prov. App. No. 62/892,724 and its PCT application, both of which are hereby incorporated by reference.

In any of the embodiments, the HEs disclosed herein may be negative for at least one, at least two, at least 3, at least 4, at least 5, or at least 6 miRNAs selected from the group consisting of miRNA-367, miRNA-302a, miRNA-302b, miRNA-302c, miRNA-223, and miRNA-142-3p. In an embodiment, the HEs are negative for miRNA-223, and miRNA-142-3p. In another embodiment, the HEs are negative for miRNA-367, miRNA-302a, miRNA-302b, miRNA-302c, miRNA-223, and miRNA-142-3p.

In an embodiment, the HEs are positive for miRNA-214, miRNA-199a-3p, and miRNA-335 (miRNA-335-5p and/or miRNA-335-3p), and negative for miRNA-223, and miRNA-142-3p.

In an embodiment, the HEs are genetically modified. The HEs may be genetically modified such that they express gene products that are believed to or are intended to promote the therapeutic response(s) provided by the cells. For example, the HEs may be genetically modified to express and/or a heterologous protein from the cells such as vascular endothelial growth factor (VEGF) and its isoforms, fibroblast growth factor (FGF, acid and basic), angiopoietin-1 and other angiopoietins, erythropoietin, hemoxygenase, transforming growth factor-α (TGF-α), transforming growth factor-β(TGF-β) or other members of the TGF-0 super family including BMPs 1, 2, 4, 7 and their receptors MBPR2 or MBPR1, hepatic growth factor (scatter factor), hypoxia inducible factor (HIF), endothelial nitric oxide synthase, prostaglandin I synthase, Krupple-like factors (KLF-2, 4, and others), and any other heterologous protein useful for promoting a therapeutic response against vascular diseases.

“Vascular disease” as used herein refers to any abnormal condition or injury of the heart, lungs, and/or blood vessels (arteries, veins, and capillaries). Vascular disease includes, but is not limited to, diseases, disorders, and/or injuries of the pericardium (i.e., pericardium), heart valves (e.g., incompetent valves, stenosed valves, rheumatic heart disease, mitral valve prolapse, aortic regurgitation), myocardium (coronary artery disease, myocardial infarction, heart failure, ischemic heart disease, angina) blood vessels (e.g., arteriosclerosis, aneurysm) or veins (e.g., varicose veins, hemorrhoids). Vascular disease, as used herein also includes, but is not limited to, coronary artery diseases (e.g., arteriosclerosis, atherosclerosis, and other diseases or injuries of the arteries, arterioles and capillaries or related complaint), myocardial infarction, (e.g. acute myocardial infarction), organizing myocardial infarct, ischemic heart disease, arrhythmia, left ventricular dilatation, emboli, heart failure, congestive heart failure, subendocardial fibrosis, left or right ventricular hypertrophy, myocarditis, chronic coronary ischemia, dilated cardiomyopathy, restenosis, arrhythmia, angina, hypertension (eg. pulmonary hypertension, glomerular hypertension, portal hypertension), myocardial hypertrophy, peripheral arterial disease including critical limb ischemia, cerebrovascular disease, renal artery stenosis, aortic aneurysm, pulmonary heart disease, cardiac dysrhythmias, inflammatory heart disease, congential heart disease, rheumatic heart disease, diabetic vascular diseases, and endothelial lung injury diseases (e.g., acute lung injury (ALI), acute respiratory distress syndrome (ARDS)). Vascular diseases may result from congenital defects, genetic defects, environmental influences (e.g., dietary influences, lifestyle, stress, etc.), and other defects or influences.

In an embodiment, the vascular disease is pulmonary hypertension (PH). Pulmonary hypertension includes pulmonary arterial hypertension (PAH), pulmonary hypertension with left heart disease, pulmonary hypertension with lung disease and/or chronic hypoxia, chronic arterial obstruction, and pulmonary hypertension with unclear or multifactorial mechanisms, such as sarcoidosis, histocytosis X, lymphangiomatosis, and compression of pulmonary vessels. See Galie et al. European Heart Journal 2016; 37(1):67-119. In a specific embodiment, the vascular disease is PAH.

Exemplary Therapeutic Uses

The HEs of the invention are useful for treating vascular diseases. Thus, the present invention provides a method of treating a vascular disease in a subject by administering to a subject a composition comprising HEs of the invention. In one embodiment, the vascular disease includes, but is not limited to, diseases, disorders, or injuries of the pericardium (i.e., pericardium), heart valves (i.e., incompetent valves, stenosed valves, rheumatic heart disease, mitral valve prolapse, aortic regurgitation), myocardium (coronary artery disease, myocardial infarction, heart failure, ischemic heart disease, angina) blood vessels (i.e., arteriosclerosis, aneurysm) or veins (i.e., varicose veins, hemorrhoids). In other embodiments, the vascular disease includes, but is not limited to, coronary artery diseases (i.e., arteriosclerosis, atherosclerosis, and other diseases of the arteries, arterioles and capillaries or related complaint), myocardial infarction, (e.g. acute myocarcial infarction), organizing myocardial infarct, ischemic heart disease, arrhythmia, left ventricular dilatation, emboli, heart failure, congestive heart failure, subendocardial fibrosis, left or right ventricular hypertrophy, myocarditis, chronic coronary ischemia, dilated cardiomyopathy, restenosis, arrhythmia, angina, hypertension, myocardial hypertrophy, peripheral arterial disease including critical limb ischemia, cerebrovascular disease, renal artery stenosis, aortic aneurysm, pulmonary heart disease, cardiac dysrhythmias, inflammatory heart disease, congenital heart disease, rheumatic heart disease, diabetic vascular diseases, and endothelial lung injury diseases (e.g., acute lung injury (ALI), acute respiratory distress syndrome (ARDS)).

In an embodiment, the vascular disease is pulmonary hypertension (PH). In a specific embodiment, the vascular disease is PAH.

The HEs of the invention may also be useful to treat the symptoms of vascular diseases. For example, the HEs may be used for treating a symptom of myocardial infarction, chronic coronary ischemia, arteriosclerosis, congestive heart failure, dilated cardiomyopathy, restenosis, coronary artery disease, heart failure, arrhythmia, angina, atherosclerosis, hypertension, critical limb ischemia, peripheral vascular disease, pulmonary hypertension, or myocardial hypertrophy. Treatment of one or more symptoms of the vascular disease may confer a clinical benefit, such as a reduction in one or more of the following symptoms: shortness of breath, fluid retention, headaches, dizzy spells, chest pain, left shoulder or arm pain, and ventricular dysfunction.

The HEs of the invention may exhibit certain properties that contribute to reducing and/or minimizing damage and promoting vascular repair and regeneration following damage. These include, among other things, the ability to synthesize and secrete growth factors stimulating new blood vessel formation, the ability to synthesize and secrete growth factors stimulating cell survival and proliferation, the ability to proliferate and differentiate into cells directly participating in new blood vessel formation, the ability to engraft damaged myocardium and inhibit scar formation (collagen deposition and cross-linking), and the ability to proliferate and differentiate into cells of the vascular lineage. In an embodiment, the HEs of the invention are capable of vascular repair. In one embodiment, the HEs contribute to post-injury progenitor cell replenishment under normal conditions. In another embodiment, the HEs of the invention are capable of homing to the site of vascular injury and facilitating re-endothelialization and preventing neointimal formation. Accordingly, the HEs of the present invention may be used to treat vascular tissue damaged due to injury or inflammation or disease.

The effects of treatment with HEs of the invention may be demonstrated by, but not limited to, one of the following clinical measures: increased heart ejection fraction, decreased rate of heart failure, decreased infarct size, decreased associated morbidity (pulmonary edema, renal failure, arrhythmias) improved exercise tolerance or other quality of life measures, and decreased mortality. The effects of cellular therapy may be evident over the course of days to weeks after the procedure. However, beneficial effects may be observed as early as several hours after the procedure, and may persist for several years.

The subject being treated with HEs of the invention according to the methods described herein will usually have been diagnosed as having, suspected of having, or being at risk for, a vascular disease. The vascular disease may be diagnosed and/or monitored, typically by a physician using standard methodologies. “Subject” and “patient” are used interchangeably herein and refers to any vertebrate, including, mammals, rodents, and non-mammals, such as non-human primates, sheep, dog, cow, chickens, amphibians, reptiles, etc. In a specific embodiment, the subject is primate. In another embodiment, the subject is a human.

In an embodiment, the methods of the invention may be practiced in conjunction with existing vascular therapies to effectively treat a vascular disease. The methods and compositions of the invention include concurrent or sequential treatment with non-biologic and/or biologic drugs. Non-limiting examples of non-biologic and/or biologic drugs include analgesics, such as nonsteroidal anti-inflammatory drugs, opiate agonists and salicylates; anti-infective agents, such as antihelmintics, antianaerobics, antibiotics, aminoglycoside antibiotics, antifungal antibiotics, cephalosporin antibiotics, macrolide antibiotics, miscellaneous β-lactam antibiotics, penicillin antibiotics, quinolone antibiotics, sulfonamide antibiotics, tetracycline antibiotics, antimycobacterials, antituberculosis antimycobacterials, antiprotozoals, antimalarial antiprotozoals, antiviral agents, anti-retroviral agents, scabicides, anti-inflammatory agents, corticosteroid anti-inflammatory agents, antipruritics/local anesthetics, topical anti-infectives, antifungal topical anti-infectives, antiviral topical anti-infectives; electrolytic and renal agents, such as acidifying agents, alkalinizing agents, diuretics, carbonic anhydrase inhibitor diuretics, loop diuretics, osmotic diuretics, potassium-sparing diuretics, thiazide diuretics, electrolyte replacements, and uricosuric agents; enzymes, such as pancreatic enzymes and thrombolytic enzymes; gastrointestinal agents, such as antidiarrheals, gastrointestinal anti-inflammatory agents, gastrointestinal anti-inflammatory agents, antacid anti-ulcer agents, gastric acid-pump inhibitor anti-ulcer agents, gastric mucosal anti-ulcer agents, H2-blocker anti-ulcer agents, cholelitholytic agent's, digestants, emetics, laxatives and stool softeners, and prokinetic agents; general anesthetics, such as inhalation anesthetics, halogenated inhalation anesthetics, intravenous anesthetics, barbiturate intravenous anesthetics, benzodiazepine intravenous anesthetics, and opiate agonist intravenous anesthetics; hormones and hormone modifiers, such as abortifacients, adrenal agents, corticosteroid adrenal agents, androgens, anti-androgens, immunobiologic agents, such as immunoglobulins, immunosuppressives, toxoids, and vaccines; local anesthetics, such as amide local anesthetics and ester local anesthetics; musculoskeletal agents, such as anti-gout anti-inflammatory agents, corticosteroid anti-inflammatory agents, gold compound anti-inflammatory agents, immunosuppressive anti-inflammatory agents, nonsteroidal anti-inflammatory drugs (NSAIDs), salicylate anti-inflammatory agents, minerals; and vitamins, such as vitamin A, vitamin B, vitamin C, vitamin D, vitamin E, and vitamin K.

Administration

As disclosed herein, HEs of the invention may be administered by several routes including systemic administration by venous or arterial infusion (including retrograde flow infusion) or by direct injection into the heart or peripheral tissues. Systemic administration, particularly by peripheral venous access, has the advantage of being minimally invasive relying on the natural perfusion of the heart and the ability of the vascular endothelial progenitors to target the site of damage. Cells may be injected in a single bolus, through a slow infusion, or through a staggered series of applications separated by several hours or, provided cells are appropriately stored, several days or weeks. Cells may also be applied by use of catheterization such that the first pass of cells through the heart is enhanced by using balloons to manage myocardial blood flow. As with peripheral venous access, cells may be injected through the catheters in a single bolus or in multiple smaller aliquots. Cells may also be applied directly to the myocardium by epicardial injection. This could be employed under direct visualization in the context of an open-heart procedure (such as a Coronary Artery Bypass Graft Surgery) or placement of a ventricular assist device. Catheters equipped with needles may be employed to deliver cells directly into the myocardium in an endocardial fashion which would allow a less invasive means of direct application.

In one embodiment, the route of delivery includes intravenous delivery through a standard peripheral intravenous catheter, a central venous catheter, or a pulmonary artery catheter. In other embodiments, the cells may be delivered through an intracoronary route to be accessed via currently accepted methods. The flow of cells may be controlled by serial inflation/deflation of distal and proximal balloons located within the patient's vasculature, thereby creating temporary no-flow zones which promote cellular engraftment or cellular therapeutic action. In another embodiment, cells may be delivered through an endocardial (inner surface of heart chamber) method which may require the use of a compatible catheter as well as the ability to image or detect the intended target tissue. Alternatively, cells may be delivered through an epicardial (outer surface of the heart) method. This delivery may be achieved through direct visualization at the time of an open-heart procedure or through a thoracoscopic approach requiring specialized cell delivery instruments. Furthermore, cells could be delivered through the following routes, alone, or in combination with one or more of the approaches identified above: subcutaneous, intramuscular, intra-tracheal, sublingual, retrograde coronary perfusion, coronary bypass machinery, extracorporeal membrane oxygenation (ECMO) equipment and via a pericardial window.

In one embodiment, cells are administered to the patient as an intra-vessel bolus or timed infusion.

Compositions

The present invention provides compositions comprising HEs. In certain embodiments, the composition comprises at least 1×10³ HEs. In another embodiment, the composition comprises at least 1×10⁴ HEs. In other embodiments, the composition comprises at least 1×10⁵, at least 1×10⁶, at least 1×10⁷, or at least 1×10⁸ HEs. The compositions may additionally comprise additives known in the art to enhance, control, or otherwise direct the intended therapeutic effect.

In an embodiment, the composition of the invention further comprises a biocompatible matrix, such as a solid support matrix, biological adhesives or dressings, or biological scaffolds, or bio-ink used for 3D bio-printing. The biocompatible matrix may facilitate in vivo tissue engineering by supporting and/or directing the fate of the implanted cells. Non-limiting examples of biocompatible matrices include solid matrix materials that are absorbable and/or non-absorbable, such as small intestine submucosa (SIS), e.g., porcine-derived (and other SIS sources); crosslinked or non-crosslinked alginate, hydrocolloid, foams, collagen gel, collagen sponge, polyglycolic acid (PGA) mesh, polyglactin (PGL) mesh, fleeces, foam dressing, bioadhesives (e.g., fibrin glue and fibrin gel), dead de-epidermized skin equivalents, hydrogels, albumin, polysaccharides, polylactic acid (PLA), polyglycolic acid (PGA), polylactic acid-glycolic acid (PLGA), polyorthoesters, polyanhydrides, polyphosphazenes, polyacrylates, polymethacrylates, ethylene vinyl acetate, polyvinyl alcohols, and the like.

The HEs of the invention may be formulated into a pharmaceutical composition comprising the HEs and a pharmaceutically acceptable carrier. Pharmaceutically acceptable carriers are well known in the art and include saline, aqueous buffer solutions, solvents, dispersion media, or any combination thereof. Non-limiting examples of pharmaceutically acceptable carriers include sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations. In an embodiment, the pharmaceutically acceptable carrier is stable under conditions of manufacture and storage. In an embodiment, the HEs of the invention are formulated in GS2 media described in WO 2017/031312, which is hereby incorporated by reference in its entirety.

The present invention further provides cryopreserved compositions comprising HEs. The cryopreserved composition may further comprise a cryopreservant. Cryopreservants are known in the art and include, but are not limited to, dimethyl sulfoxide (DMSO), glycerol, etc. The cryopreserved composition may also comprise an isotonic solution, such as a cell culture medium.

The present invention is further illustrated by the following examples, which are not intended to be limiting in any way. The entire contents of all references, patents and published patent applications cited throughout this application, as well as the Figures, are hereby incorporated herein by reference.

### EXAMPLES

### Example 1: Generation of Hemogenic Endothelial Cells (HEs)

Hemogenic endothelial cells were generated from human embryonic stem cells (hESCs) or human induced pluripotent stem cells (iPSCs) as shown in FIG. 1. hESCs (eg. J1 hESCs) or iPSCs (eg. GMP-1 iPSCs) were cultured for four days in mTeSR1 (Stemcell Technology) plus 1% penicillin/streptomycin on human dermal fibroblast feeder cells in 6 well plates and the media was changed daily. To plate the hESCs or iPSCs for differentiation (Day −1), the mTeSR1 medium was removed from each well of the 6 well plate. Each well was washed with 2 mL of DMEM/F12 (Gibco) or D-PBS, the DMEM/F12 or D-PBS aspirated, and 1 mL of enzyme-free Gibco® Cell Dissociation Buffer (CDB) was added to each well. The plate was incubated inside a normoxic CO₂ incubator (5% CO₂/20% O₂) for about 5-8 minutes until the cells showed a detached morphology. CDB was then carefully removed by pipetting without disturbing loosely attached cells. The cells were collected by adding 2 mL of mTeSR1 to each well and collected in collection tubes. The remaining cells in the wells were washed gently with an additional 2 mL mTeSR1 and transferred to the collection tubes. The tubes were centrifuged at 120×g for 3 min and the culture medium was removed. The cells were resuspended at a final density of 400,000 cells/10 mL in mTeSR1 medium containing Y-27632 (Stemgent) at a final concentration of 10 μM. 10 mL of the cell suspension was transferred into a collagen IV-coated 10 cm plate. The plates were placed in the normoxic incubator overnight.

The next day (Day 0), the mTeSR1/Y-27632 media was gently removed from each 10 cm plate and replaced with 10 mL of BVF-M media [Stemline® II Hematopoietic Stem Cell Expansion Medium (Sigma); 25 ng/mL BMP4 (Humanzyme); 50 ng/mL VEGF165 (Humanzyme); 50 ng/mL FGF2 (Humanzyme)]. The plates were incubated in a hypoxia chamber (5% CO₂/5% O₂) for 2 days.

On Day 2, the media was aspirated and fresh 10-12 mL of BVF-M was added to each 10 cm plate.

On Day 4, the media was again aspirated and fresh 10-15 mL of BVF-M was added to each 10 cm plate.

On Day 6, the cells were harvested for transplantation and/or for further testing. The media was aspirated from each plate and the plates were washed by adding 10 mL of D-PBS (Gibco) and aspirating the D-PBS. 5 mL of StemPro Accutase (Gibco) was added to each 10 cm plate and incubated for 3-5 min in a normoxic CO₂ incubator (5% CO₂/20% O₂). The cells were pipetted 5 times with a 5 mL pipet, followed by a P1000 pipet about 5 times. The cells were then strained through a 30 μM cell strainer and transferred into a collection tube. Each of the 10 cm plates were again rinsed with 10 mL of EGM2 medium (Lonza) or Stemline® II Hematopoietic Stem Cell Expansion Medium (Sigma) and the cells were passed through a 30 μM cell strainer and collected in the collection tube. The tubes were centrifuged at 120-250 g for 5 min. The cells were then resuspended with EGM2 media or or Stemline® II Hematopoietic Stem Cell Expansion Medium (Sigma) and counted. After counting, the cells were spun down (250×g for 5 min) and resuspended with with Freezing medium (10% DMSO+Heat Inactivated FBS) in concentration of 3×10⁶ cells/mL. To create frozen stocks, cell suspension was aliquoted in 2 mL FBS (Hyclone) and DMSO (Sigma) per cryovial (6×10⁶ cells/2 mL/vial).

### Example 2: Generation of Hemangioblasts (HB)

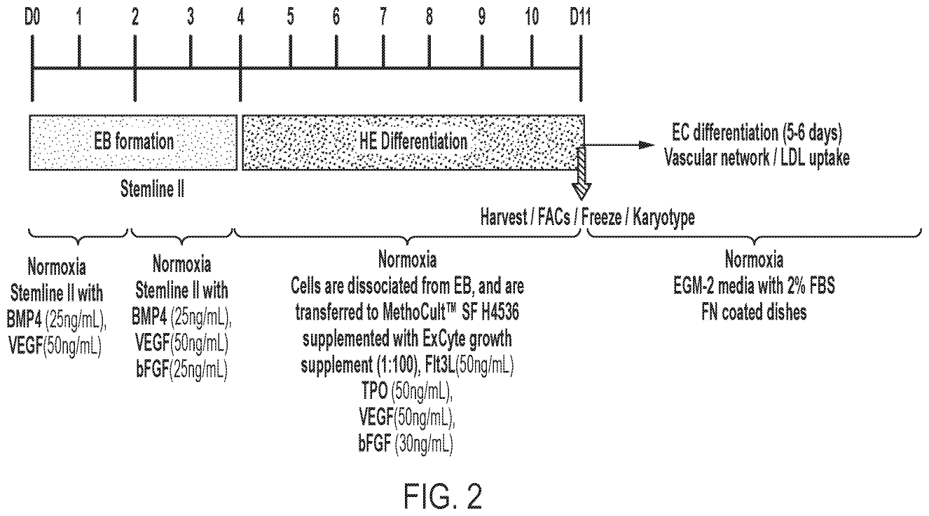

Hemangioblasts were generated from human embryonic stem cells (eg. J1 hESCs) or human induced pluripotent stem cells (eg. GMP-1 iPSCs) as shown in FIG. 2. hESCs or iPSCs cultured in mTeSR1 (Stemcell Technology) plus 1% penicillin/streptomycin on human dermal fibroblast feeder cells in 6 well plates were lifted off the wells by incubating each well with DMEM/F12 (Gibco) containing 4 mg/mL collagenase IV (Gibco) for about 10 min at 37° C. (5% CO₂/20% O₂) in an incubator until cells detached from the plate. The DMEM/F12 containing collagenase IV was removed from each well, washed with DMEM/F12, and 2 mL mTeSR1 was added to each well and a cell scraper was used, when necessary, to detach cells from the wells. The cell suspension was transferred to a conical tube and each well was washed again with 2 mL of mTeSR1 and transferred to the conical tube. The tube was centrifuged at 300×g for 2 min and the supernatant was removed. The cell pellet was resuspended in BV-M media [Stemline® II Hematopoietic Stem Cell Expansion Medium (Sigma); 25 ng/mL BMP4 (Humanzyme); 50 ng/mL VEGF165 (Humanzyme)] and plated onto Ultra Low Attachment Surface 6 well plates (Corning) at a density of about 750,000-1,200,000 cells per well. The plates were placed in an incubator for 48 hrs in a normoxic CO₂ incubator to allow embryoid body formation (Days 0-2). The media and cells in each well were then collected and centrifuged at 120-300 g for 3 min. Half of the supernatant was removed and replaced with 2 mL BV-M containing 50 ng/mL bFGF. Therefore, the final concentration of bFGF in the cell suspension was about 25 mg/mL 4 mL of the cell suspension was plated onto each well of a Ultra Low Attachment Surface 6 well plates and placed into a normoxic CO₂ incubator for another 48 hrs (Days 2-4) to allow continued embryoid body formation.

On Day 4, the embryoid bodies were collected into a 15 mL tube, centrifuged at 120-300×g for 2 min, washed with D-PBS, and disaggregated into single cell suspensions using StemPro Accutase (Gibco). FBS (Hyclone) was used to inactivate the Accutase and the single cells were passed through a cell strainer, centrifuged, and resuspended in Stemline II media (Sigma) at about 1×10⁶ cells/mL. About 3×10⁶ cells were mixed in 30 mL Methocult BGM medium [MethoCult™ SF H4536 (no EPO) (StemCell Technologies); penicillin/streptomycin (Gibco); ExCyte Cell Growth Supplement (1:100) (Millipore); 50 ng/mL Flt3 ligand (PeproTech); 50 ngm/ml VEGF (Humanzyme); 50 ng/mL TPO (PeproTech); 30 ng/mL bFGF (Humanzyme)], replated on Ultra Low Attachment Surface 10 cm dishes (Corning), and incubated in a normoxic CO₂ incubator for 7 days (Days 4-11) to allow for formation of hemangioblasts.

On Day 11, the hemangioblasts were harvested for transplantation and/or for further testing. Hemangioblasts were collected by diluting the methylcellulose with D-PBS (Gibco). The cell mixture was centrifuged at 300×g for 15 min twice, and resuspended in 30 mL of EGM2 BulletKit media (Lonza) or StemlineII and the cells were counted and frozen as described above.

### Example 3: Cell Marker Analysis

HEs harvested at Day 6 according to Example 1 and hemangioblasts (HB) harvested on Day 11 according to Example 2 were analyzed for endothelial cell markers, blood/hemogenic markers, and pericyte markers by FACS analysis. Briefly, the harvested cells were resuspended in 50 uL of FACS buffer (2% FBS/PBS) at a density of 100 k/tube. The flow cytometry antibodies were added according to Table 1 and incubated for 20 minutes at 4° C. 1 mL of FACS buffer was then added to each tube and centrifuged for 5 minute at 250×g. The cells were resuspended in 200 uL of FACS buffer without propidium iodide (PI) per tube. The samples were analyzed on MACS Quant Analyzer 10 (Miltenyi Biotec: 130-096-343). HUVEC were used for positive and HDF or undifferentiated hESCs were used as negative control. In addition, HUVEC was used as single staining (SS) control for compensation.

TABLE 1
Antibody Staining Table for MACS Quant Analyzer 10

| | FITC/ | | | | |
|---|---|---|---|---|---|
| | AF488 | PE | APC | APC-Vio770 | Vio Blue |
| Stain1 | CD43 | CD34 | FLK1 | CXCR4 | CD31 |
| | (1:50) | (1:50) | (1:50) | (1:50) | (1:100) |
| Stain2 | CD146 | cKit | CD144 | Tie2 | CD31 |
| | (1:50) | (1:50) | (1:50) | (1:50) | (1:100) |
| Stain3 | CD105 | CD31 | CD271 | CD44 | CD274 |
| | (1:50) | (1:100) | (1:100) | (1:50) | (1:50) |
| Stain4 | CD90 | NG2 | CD140b | VCAM1 | CD31 |
| | (1:50) | (1:25) | (1:50) | (1:50) | (1:100) |
| SS-1 | CD31 | | | | |
| | (1:100) | | | | |
| SS-2 | | CD31 | | | |
| | | (1:100) | | | |
| SS-3 | | | CD31 | | |
| | | | (1:100) | | |
| SS-4 | | | | CD31 | |
| | | | | (1:100) | |
| SS-5 | | | | | CD31 |
| | | | | | (1:100) |
| Unstained | | | | | |

Alternatively, FACs analysis was performed using a SONY SA3800 Spectral Analyzer. Briefly, the harvested cells were resuspended in 100 uL of FACS buffer (2% FBS/PBS) at a density of 100-200 k/tube. The flow cytometry antibodies were added according to Table 2 and incubated for 20 min at 4° C. 1 mL of FACS buffer was then added to each tube and centrifuged for 5 min at 300×g. The cells were resuspended in 1004, FACS buffer with or without PI (1:1000 dilution with FACS buffer) per tube. The samples were analyzed on a SONY SA3800 Spectral Analyzer. HUVEC cells were used as a positive control and undifferentiated hESCs were used as a negative control.

TABLE 2
Antibody Staining Table for SONY SA3800 Spectral Analyzer

| Tube # | | FITC | PE | APC | PI |
|---|---|---|---|---|---|
| 1 | Unstained | − | − | − | − |
| 2 | PI only | − | − | − | + |
| 3 | CD31 FITC | + | − | − | − |
| 4 | CD31 PE | − | + | − | − |
| 5 | CD31 APC | − | − | + | − |
| 6 | Staining 1 | CD34 | CD31 | CD144 | + |
| 7 | Staining 2 | CD43 | CD45 | CD184 | + |
| 8 | Staining 3 | CD146 | NG2 | PDGFRb | + |
| 9 | Staining 4 | CD146 | CXCR7 | CD309 | + |

Results

As shown in Tables 3-4, the HBs were positive for both blood markers CD43 and CD45 and endothelial cell markers CD31, CD144 & CD34 but expressed low or undetectable levels of Tie2, CD140b, CD90, and CD271.

In contrast, as shown in Tables 3-4, the HEs were positive for CD146, CXCR4 and Flk1 (CD309/KDR) as well as pericyte/mesenchymal markers CD90 and CD271 but were negative for the blood/hemogenic markers CD43 and CD45.

TABLE 3
Summary of cell surface markers on HBs and HEs derived
from J1 and GMP1 lines and analyzed on the MACS Quant
Analyzer 10 and/or SONY SA3800 Spectral Analyzer).

| | Markers | HE | HB |
|---|---|---|---|
| Endothelial | CD31/PECAM1 | 20-50% | 75-99% |
| markers | | (n = 12) | (n = 17) |
| | CD309/KDR | 10-50% | 1-15% |
| | | (n = 9) | (n = 10) |
| | CD144/VE-cadh | 5-40% | 15-60% |
| | | (n = 12) | (n = 17) |
| | CD34 | 5-20% | 10-50% |
| | | (n = 12) | (n = 17) |
| Chemokine | CXCR4/CD184 | 20-60% | 10-20% |
| receptors | | (n = 12) | (n = 17) |
| Chemokine | CXCR7 | 4-10% | Less than 5% |
| receptors | | (n = 9) | (n = 10) |
| Blood | CD43 | Less than 10% | 70-95% |
| markers | | (n = 12) | (n = 17) |
| Blood | CD45 | Less than 5% | 50-90% |
| markers | | (n = 9) | (n = 10) |
| Pericyte | CD146 | 50-95% | 5-20% |
| markers | | (n = 9) | (n = 10) |

TABLE 4
Summary of cell surface markers on J1-derived
HB and HE analyzed on MACS Quant Analyzer 10.

| | J1-HE | | | |
|---|---|---|---|---|
| | (n = 3) | | J1-HB | |
| | Frequency (%) | SD (%) | Frequency (%) | SD (%) |
| Tie2 | 35.0 | 0.76 | 0.9 | 0.50 |
| CD140b | 27.7 | 9.42 | 0.3 | 0.14 |
| CD90 | 37.5 | 9.09 | 0.7 | 0.21 |
| CD271 | 30.2 | 12.62 | 0.1 | 0 |

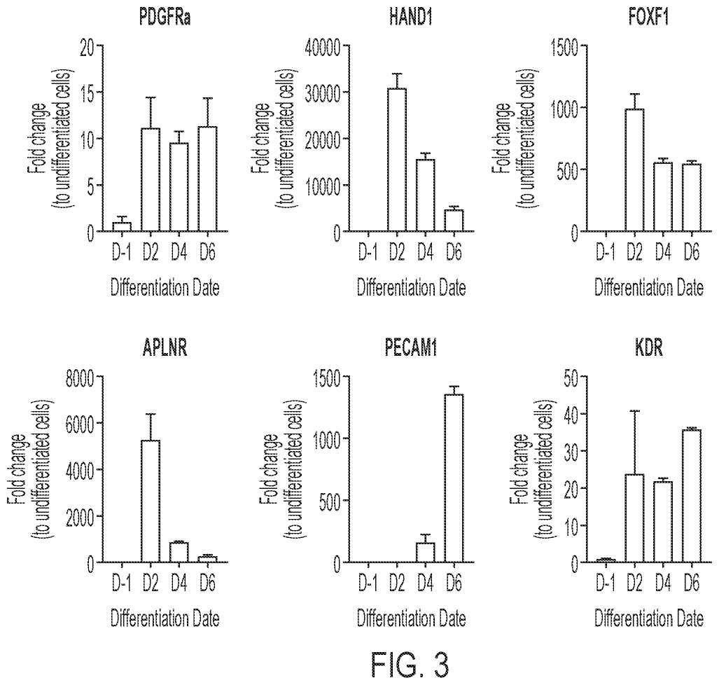

A time course of cell marker expression in various cells during the differentiation process showed that cells upregulated markers of the mesodermal lineage, with surface expression of PDGFRA and APLNR peaking at day 2. Subsequently, expression of those markers declined, which correlated with an increase in CD31, a marker of vascular cells, at day 6 (FIG. 3). Examination by light microscopy suggested that the differentiation method generated a mixture of cells, with cells displaying endothelial or mesenchymal morphologies.

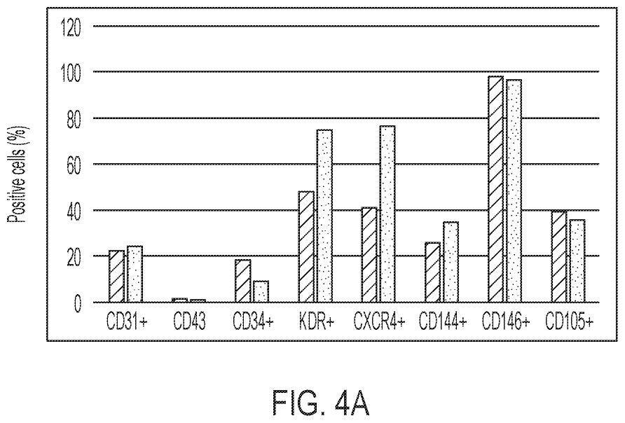

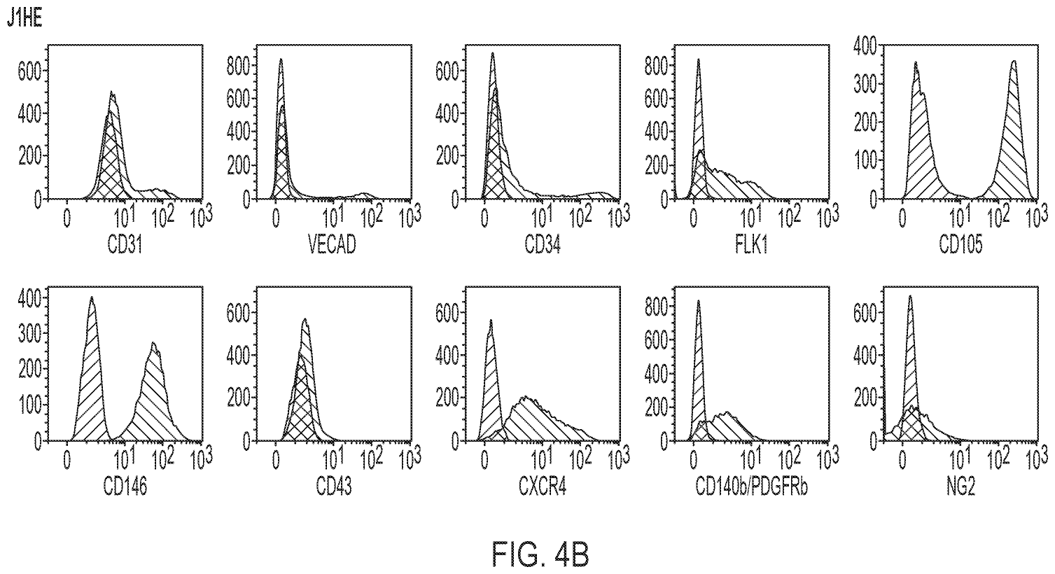

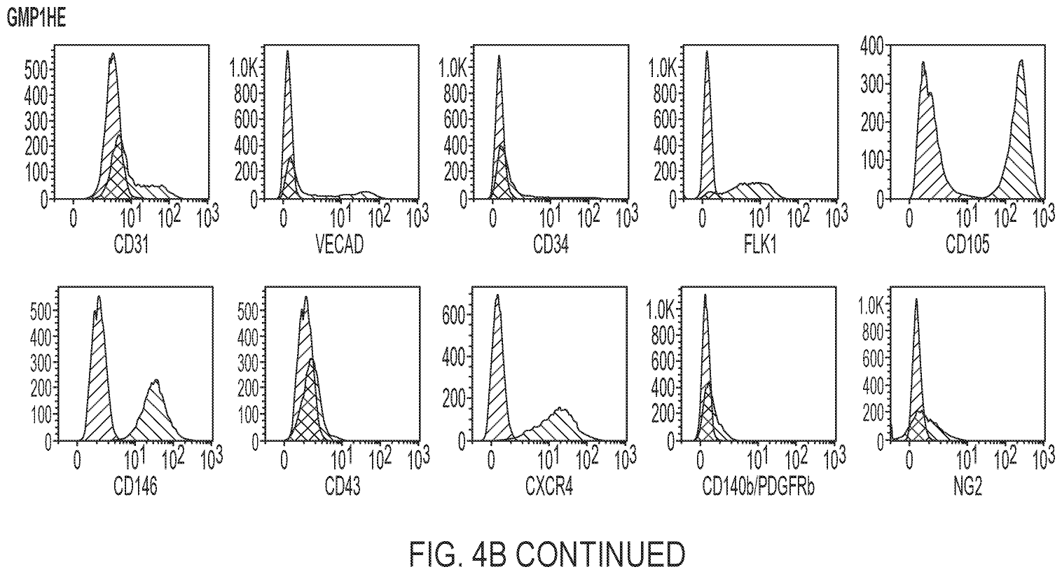

Further characterization of the HE cells produced at day 6 showed that the majority of the cells were CD146+ expressing either VECAD+(CD144+) or CD140B+(PDGFRB+) but no hematopoietic markers CD43 and CD45, indicating that the protocol produced distinct vascular and perivascular cells. Additional characterization of the HE cells produced at day 6 was performed for CD31, CD43, CD34, KDR (FLK1), CXCR4, CD144, CD146, CD105, CD140b (PDGFRb), and NG2 and are shown in FIGS. 4A and 4B.

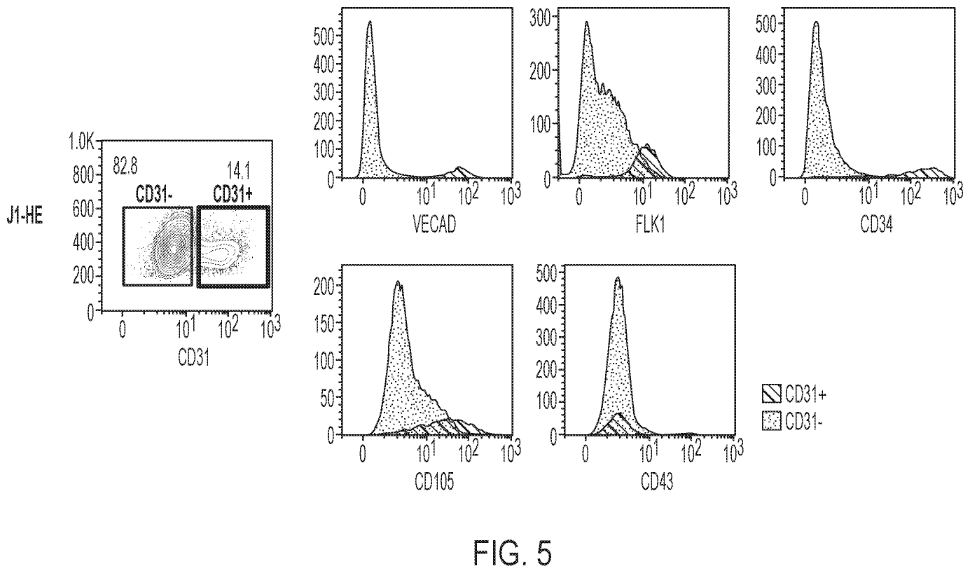

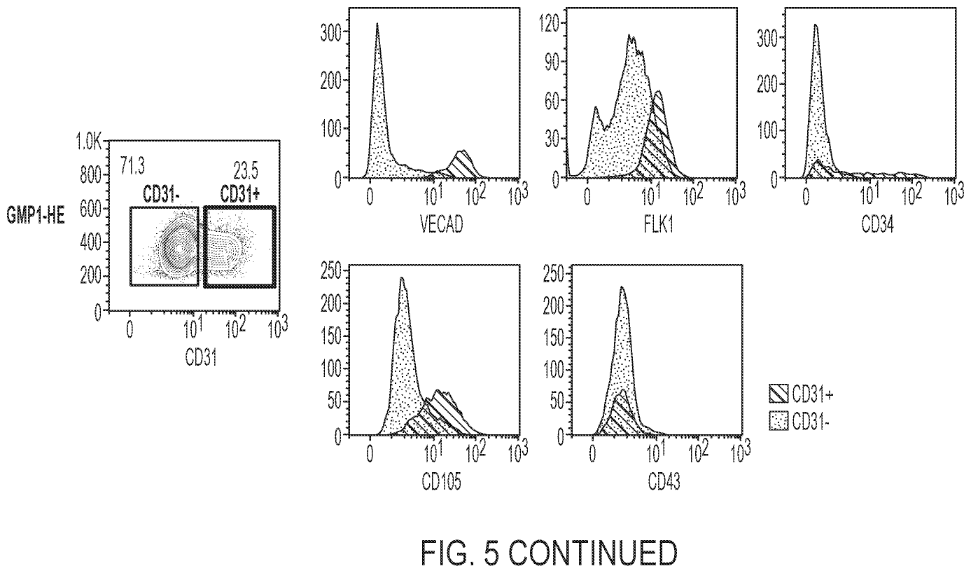

A presumptive vascular endothelial fraction, identified by CD31 expression, was positive for FLK1/CD309 [also known as VEGFR2], VECAD, CD34, and CD105 (FIG. 5). When day 6 HE cells were transferred to medium supportive of vascular endothelial cell growth for an additional 5-7 days in normoxic conditions, CD31, CD34, and FLK1/CD309 (VEGFR2) expression was maintained or increased.

### Example 4: HEs Express Endothelial, Smooth Muscle, and Pericyte Markers

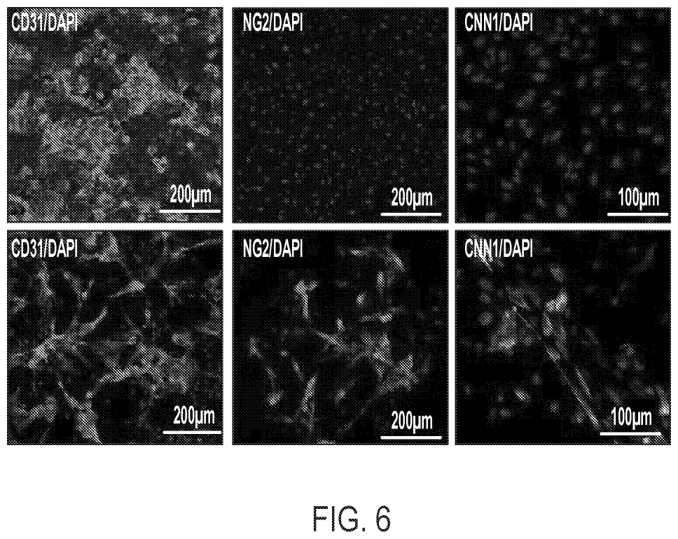

Additional analysis using immunocytochemistry (ICC) was performed as described below using HUVEC as control. HEs were plated for at least 24 h and then washed with D-PBS with Ca2+ and Mg2+(Gibco) twice. Then cells were fixed with 4% PFA (Electron Microscopy Science) for 10 minutes at room temperature. After fixations, cells were washed with D-PBS with Ca2+ and Mg2+ for 5 minutes three times. The cells were then treated with 1× Perm/Wash buffer (BD) containing 5% normal goat serum (Cell Signaling Technology) for one hour. After aspiration of Perm/Wash/Blocking buffer, cells were treated with primary antibody containing Perm/Wash/Blocking buffer (human CD31, 1:50, Invitrogen; human NG2, 1:50, PD Pharmagen; human Calponin, 1:100, Millipore) overnight. Next day, cells were washed with Perm/Wash buffer 5 minutes three times. Cell were then treated with secondary and DAPI containing Perm/Wash/Blocking buffer (DAPI, 1:1000, Invitrogen; Goat-anti Ms-Cy3, Goat-anti Rb-Alexaflour488) for 1 hour at room temperature. Cells were washed three times for 5 minutes with Perm/Wash buffer and images were captured with Keyence BZ-X710 (Keyence). As shown in FIG. 6, HEs expressed endothelial (CD31), smooth muscle (Calponin) and pericyte markers (NG2) and therefore have the capacity to differentiate into endothelial cells, smooth muscle cells, and pericytes. Additionally, when day 6 HE cells were transferred to medium supportive of pericyte cell growth, CD140B expression slightly decreased and NG2, CD90, CD73, CD44, and CD274 expression was maintained or increased (data not shown).

### Example 5: Single Cell miRNA Profile

Additional analysis using single cell qRT-PCR analysis to evaluate the levels of expression of 96 microRNA associated with pluripotency or vascular cell identity was performed as described below on II-derived HB and HE. TaqMan Gene Expression Assays (Applied Biosystems) were ordered for 96 human miRNAs. 10× Assays were prepared by mixing 25 μL of 20× Taqman assays with 25 μL of 2× Assay Loading Reagent (Fluidigm) for a 50 μL volume of final stock. An aliquot of cells (frozen or freshly harvested) in the range of 66,000 to 250,000 cells/mL was prepared. The cells were incubated with LIVE/DEAD staining solution (LIVE/DEAD Viability/Cytotoxicity Kit) for 10 minutes at room temperature. The cells were then washed, suspended in media and filtered through a 40 μm filter. Cell counting was performed for viability and cell concentration using cellometer. A cell mix was prepared by mixing cells (60 μL) with suspension reagent (40 μL) (Fluidigm) in a ratio of 3:2. 6 μL of the cell suspension mix was loaded onto a primed C1 Single-Cell Autoprep IFC microfluidic chip for medium cells (10-17 μm) or large cells (17-25 μm), and the chip was then processed on the Fluidigm C1 instrument using the “STA: Cell Load(1782×/1783×/1784×)” script. This step captured one cell in each of the 96 capture chambers. The chip was then transferred to a Keyence Microscope and each chamber was scanned to score number of single cell captures, live/dead status of cells and doublet/cell aggregates captured. For Cell Lysis, Reverse Transcription, and Preamplification on the C1, Harvest reagent, Lysis final mix, RT final mix and Preamp mix were added to designated wells of the C1 chip according to manufacturer's protocol. The IFC was then placed in the C1 and “STA:miRNA Preamp (1782×/1783×/1784×) script was used. The cDNA harvest was programmed to finish the next morning. The cDNA was transferred from each chamber of the C1 chip to a fresh 96 well plate that was pre-loaded with 12.5 μL of C1DNA dilution reagent. Tube controls such as the no template control and the positive control were prepared for each experiment according to manufacturer's instructions. Preamplified cDNA samples were analyzed by qPCR using the 96.96 Dynamic Array™ IFCs and the BioMark™ HD System. Processing of the IFC priming in JUNO instrument followed by loading of cDNA sample mixes and 10× Assays was performed per manufacturer's protocol. The IFC was then placed into the Biomark™ HD system and PCR was performed using the protocol “GE96×96 miRNA Standard v1.pcl”. Data analysis was performed using the Real-Time PCR Analysis software provided by Fluidigm. The dead cells, duplicates etc were removed from analysis and the Linear Derivative Baseline and User Detector Ct Threshold based methods were used for analysis. The data was viewed in Heatmap view and exported as a CSV File. “R” software was then used to perform “Outlier Identification” analysis that resulted in a “FSO” file, and then instructions for “Automatic Analysis” were followed.

TABLE 5
miRNA marker profile

| | | | |
|---|---|---|---|
| J1 | J1-HE | J1-HB | HUVEC |

Pluripotent miRNA

| | | | | |
|---|---|---|---|---|
| 367 | + | − | − | − |
| 302 a | + | − | − | − |
| 302 b | + | − | − | − |
| 302 c | + | − | − | − |

Vascular miRNA

| | | | | |
|---|---|---|---|---|
| 126 | − | + | + | + |
| 24 | − | + | + | + |
| 196-b | − | + | − | + |

Unique miRNA

| | | | | |
|---|---|---|---|---|
| 223 | − | − | + | − |
| 142-3p | − | − | + | − |
| 214 | − | + | − | − |
| 199a-3p | − | + | − | − |
| 335 | − | + | − | − |

Results

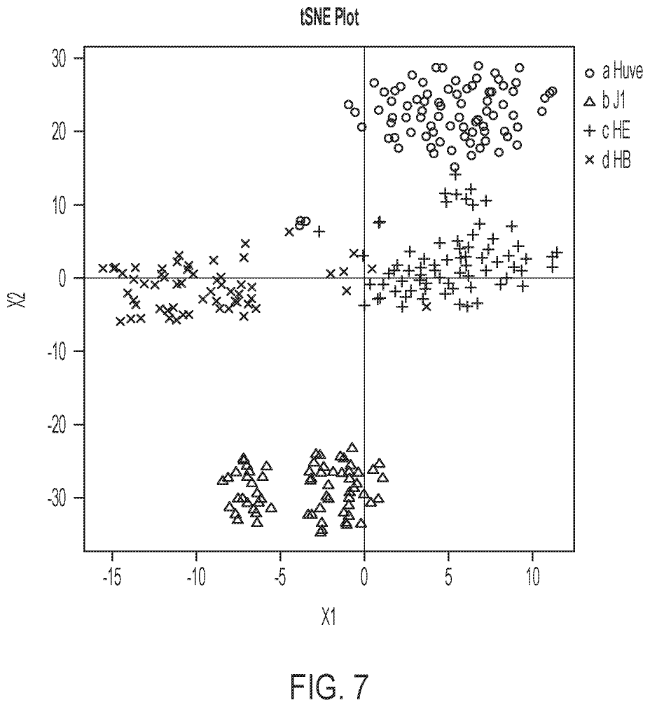

As shown in FIG. 7, J1-HE cells had a distinct miRNA expression profile compared to undifferentiated embryonic stem cells (J1), human vascular cells (HUVEC) and J1-derived HB cells. Specific examples of miRNA markers are shown in Table 5.

### Example 6: In Vitro Differentiation into Endothelial Cells and Vascular Tube Formation

HEs and HBs derived from J1 and GMP-1 were further tested in vitro for their ability to differentiate into endothelial cells. Approximately 300 k of the HE cells and 500-600 k of the HBs were resuspended in 18 mL of EGM2 or Vasculife VEGF medium kit (Lifeline Cell Tech) and 3 mL of the resuspension was aliquoted into each well of a fibronectin-coated 6 well plate (Corning). After two days in culture, the medium was changed and fresh EGM2 or Vasculife VEGF medium was added. Pictures were taken when cells reached about 60-70% confluence. HBs (at day 5) and HEs (at day 3) differentiated towards the endothelial lineage in fibronectin-coated plates and both showed characteristic endothelial cobblestone morphology (data not shown).

To test for vascular tube formation, cells were harvested after pictures were taken. Briefly, each well was washed with D-PBS and 1 mL StemPro Accutase (Gibco) was added to each well and incubated for 3-5 min at 37° C. A single cell suspension was generated by pipetting the culture a few times. The plate was washed with EGM2 medium or Vasculife VEGF medium and transferred to a conical tube and centrifuged at 250 g for 5 min. A cell count was performed using the Nexcelom Cellometer K2.

250 μL basement membrane Matrigel (Corning) was added to each well of Nunc™ 4 well plates (Thermo Scientific) and the plates were incubated for 30 min at RT. Harvested HBs and HEs were seeded at a density of about 5.0×10⁴ cells in 250 μL EGM2 media or Vasculife VEGF media per well. After 2-3 hours of plating, the media were replaced with fresh 250 μL media containing AcLDL (Molecular Probes) (5 μL AcLDL plus 245 μL media). Plates were then incubated overnight in a normoxia condition. After 24 hours of incubation, AcLDL-containing media were removed, the plates were washed with D-PBS 3 times, and fresh 250 μL EGM2 medium or Vasculife VEGF medium/well was added. Finally, photomicrographs were taken from each well at 4× magnification using a Keyence Microscope. Both HBs and HEs formed vascular-like networks on Matrigel (data not shown).

### Example 7: In Vivo Study in a Pulmonary Arterial Hypertension Model

The purpose of this study was to assess the effect of the hemogenic endothelial cells on the Sugen-Hypoxia (SuHx)-induced pulmonary arterial hypertension (PAH) in rats. The study also evaluated the potential efficacy of hemogenic endothelial cells for the treatment of SuHx induced pulmonary hypertension (PAH) in nude rats. The SuHx-induced pulmonary hypertension in rats is a well-documented model and is useful to investigate the effects of antihypertensive agents on pulmonary arterial pressure and right ventricular remodeling in rats with pulmonary hypertension.

Species

Male Nude (RNU) rats (Charles River Laboratories) weighing between 200 and 250 g at the time of their enrollment in the study.

Test Articles

VPC1=J1-HBs as prepared above in Example 2

VPC2=J1-HEs as prepared above in Example 1

Vehicle (Negative Control)

Distilled sterile water

Preparation of Sugen Solution

A solution of Sugen at 10 mg/mL in DMSO was prepared for administration on day 0.

Experimental Procedures

The animals were randomized in terms of even distribution between treatment groups based on their body weight.

Animals from Groups 2 to 8 (see Table 6) were subjected to the sugen/hypoxia/normoxia protocol for 21 days. Animals from Group 1 received injection of DMSO (vehicle for sugen) and subjected to hypoxia/normoxia using the same protocol. The animals were observed on a daily basis for any changes in their behavior and general health status.

Treatment with the test article or vehicle was administered at Day 1 or Day 9 as scheduled and described in Table 6. Food and water were given ad libitum. Daily observation of the behavior and general health status of the animals was done. Weekly body weights were noted.

On the day of surgery, the rats were anaesthetized with a mixture of 2 to 2.5% isoflurane USP (Abbot Laboratories, Montreal Canada) in oxygen. Hemodynamic and functional parameters (systemic arterial blood pressure, right ventricular blood pressure, pulmonary arterial blood pressure, oxygen saturation and heart rate) were recorded continuously for 5 minutes or until loss of pulmonary arterial pressure signal, whichever came first.

The rats were then exsanguinated and the pulmonary circulation was flushed with 0.9% NaCl. The lungs and heart were removed all together from the thoracic cavity. The lung (left lobe) was inflated with 10% NBF. The left lobes were prepared on slides for histopathology analysis. The hearts were excised to measure the wet weights of the right ventricle and left ventricle including the septum as part of the Fulton index.

TABLE 6
Treatment Group Assignment and Treatment Information

| | | | | Treatment | | Gr. |
|---|---|---|---|---|---|---|
| Gr. | Group | Treatment | Route of | Injection | Surgery | Size |
| # | Description | Dose | Administration | Day | Day | (n=) |
| 1 | Normoxic Control | n/a | n/a | n/a | Day 21 | 5 |
| 2 | SuHx + vehicle | n/a | i.v. jugular | Day 1 | Day 21 | 8 |
| | | | vein injection | | | |
| 3 | SuHx + VPC1 | 2.5M cells | i.v. jugular | Day 1 | Day 21 | 8 |
| | | | vein injection | | | |
| 4 | SuHx + VPC2 | 2.5M cells | i.v. jugular | Day 1 | Day 21 | 8 |
| | | | vein injection | | | |
| 5 | SuHx + VPC1 | 5.0M cells | i.v. jugular | Day 1 | Day 21 | 8 |
| | | | vein injection | | | |
| 6 | SuHx + VPC1 | 2.5M cells | i.v. jugular | Day 9 | Day 21 | 8 |
| | | | vein injection | | | |
| 7 | SuHx + VPC2 | 2.5M cells | i.v. jugular | Day 9 | Day 21 | 8 |
| | | | vein injection | | | |
| 8 | SuHx + VPC1 | 5.0M cells | i.v. jugular | Day 9 | Day 21 | 8 |
| | | | vein injection | | | |

Data Analysis

Heart rate. Heart rate was measured via a N-595 pulse oxymeter (Nonin, Plymouth, Minn.) attached to the left front paw of the animal. The heart rate values derived from the pulse oxymeter were measured in beat per minutes (bpm) using cursor readings in Clampfit 10.2.0.14 (Axon Instrument Inc., Foster City, Calif., USA, [now Molecular Devices Inc.]).

Saturation (SO₂). Blood oxygen saturation (SO₂) was read off of the pulse oxymeter (Nonin, Plymouth, Minn.) signal attached to the left front paw of the animal. The saturation values were measured in percentage (%) using cursor readings in Clampfit 10.2.0.14.

Arterial blood pressures. Arterial blood pressure was recorded continuously throughout the experiment via an intra-arterial fluid-filled catheter (AD Instruments, Colorado Springs, Colo., USA). Diastolic and systolic pressures values were measured in mmHg using cursors readings in the Clampfit 10.2.0.14. Mean arterial blood pressure values were calculated using the following formula:

Mean Arterial Pressure=Diastolic Pressure+Systolic Pressure—Diastolic Pressure)/3

Pulse pressure was calculated as the difference between systolic and diastolic readings.

Ventricular and pulmonary blood pressures. Right ventricular and pulmonary blood pressures were recorded via an intra-ventricular fluid-filled catheter (AD Instruments, Colorado Springs, Colo., USA). Diastolic and systolic pressures values were measured in mmHg using cursor readings in Clampfit 10.2.0.14. Mean ventricular and pulmonary blood pressure values were calculated using the following formula:

Mean Ventricular or Pulmonary Pressure=Diastolic Pressure+(Pulse Pressure/3)

Fulton's index. At the end of the physiological recording, the lungs and heart of each animal were removed. The heart was dissected to separate the right ventricle from the left ventricle with septum, and weighed separately. Fulton's index was then calculated using the following formula:

$$Fulton ' s index = \frac{right ventricular weight}{lef t ventricular + septum weight}$$

Statistical analysis. Values are presented as means±SEM (standard error of the means). Single-factor ANOVAs and repeat unpaired Student's t-tests were performed in Microsoft Excel 2007 on all experimental conditions, comparing treatment groups to either the control, healthy animals, or the Sugen-Hypoxia animals (vehicle). Differences were considered significant when p≤0.05.

Throughout the results, * indicates that the value is significantly different from the normoxic control group (Group 1) while ** indicates that the value is significantly different from the SuHx control group (Group 2). In other words, * indicates that the animals are significantly different from healthy animals, while ** indicates that the animals are significantly different from fully sick animals who have not benefited from any therapeutic treatment.

Results

The Sugen+Hypoxia (SuHx)-induced PAH rat model is a widely used model to study pulmonary arterial hypertension. Sugen is a VEGF-receptor antagonist known to cause pulmonary endothelial lesions, initially damaging approximately 50% of the endothelial cells in the pulmonary vasculature at the exposure level used in this study (single dose of 20 mg/kg). Remodeling of the damaged endothelial and vascular cells as well as vasoconstriction occur and obstruct the pulmonary arterioles, thus limiting the blood flow through the pulmonary arteries and increasing pulmonary arterial pressure. The decrease in blood flow through the pulmonary arteries and the increase of the pulmonary arterial pressure increase the right ventricular afterload, leading to the development of a marked right ventricular hypertrophy characteristic of SuHx-treated rats, and observed in clinical patients suffering from PAH.

In this study, all SuHx+vehicle only animals developed a medium to severe PAH as expected. The diseased animals presented all the characteristics of the PAH model: The pulmonary pressures (systolic, diastolic and mean) were statistically higher in the SuHx animals compared to healthy animals (Tables 7, 8, and 9). With a value of 41.2 mmHg (Table 9), the mean pulmonary pressure was 3-fold higher in the SuHx+vehicle animals than in healthy animals, corresponding to the higher range of medium/severe luminary arterial hypertension.

TABLE 7
Effect of VPC1 and VPC2 on systolic pulmonary
pressure of sugen-hypoxia induced PAH rate.

| | Systolic | | | |
|---|---|---|---|---|
| | Pulmonary | | | |
| | Pressure | | | |
| Treatment | (mmHg) | SEM | p value | n= |
| Normoxic Control | 24.0 | 1.55 | n/a | 5 |
| Vehicle | 66.6* | 6.01 | 0.000 | 8 |
| VPC1, 2.5M cells injected at Day 1 | 55.3 | 5.45 | 0.183 | 8 |
| VPC2, 2.5M cells injected at Day 1 | 51.7 | 5.33 | 0.090 | 7 |
| VPC1, 5M cells injected at Day 1 | 59.9 | 4.75 | 0.388 | 9 |
| VPC1, 2.5M cells injected at Day 9 | 62.5 | 5.03 | 0.625 | 6 |
| VPC2, 2.5M cells injected at Day 9 | 60.2 | 4.80 | 0.410 | 10 |
| VPC1, 5M cells injected at Day 9 | 62.4 | 3.69 | 0.540 | 10 |

TABLE 8
Effect of VPC1 and VPC2 on diastolic pulmonary
pressure of sugen-hypoxia induced PAH rat

| | Diastolic | | | |
|---|---|---|---|---|
| | Pulmonary | | | |
| | Pressure | | | |
| Treatment | (mmHg) | SEM | p value | n= |
| Normoxic Control | 8.6 | 0.60 | n/a | 5 |
| Vehicle | 28.5* | 2.35 | 0.000 | 8 |
| VPC1, 2.5M cells injected at Day 1 | 23.5 | 2.64 | 0.179 | 8 |
| VPC2, 2.5M cells injected at Day 1 | 21.6** | 0.90 | 0.022 | 7 |
| VPC1, 5M cells injected at Day 1 | 28.6 | 1.91 | 0.985 | 9 |
| VPC1, 2.5M cells injected at Day 9 | 25.5 | 0.85 | 0.310 | 6 |
| VPC2, 2.5M cells injected at Day 9 | 26.2 | 1.93 | 0.455 | 10 |
| VPC1, 5M cells injected at Day 9 | 26.5 | 1.38 | 0.452 | 10 |

TABLE 9
Effect of VPC1 and VPC2 on mean pulmonary
pressure of sugen-hypoxia induced PAH rat

| | Mean | | | |
|---|---|---|---|---|
| | Pulmonary | | | |
| | Pressure | | | |
| Treatment | (mmHg) | SEM | p value | n= |
| Normoxic Control | 13.7 | 0.64 | n/a | 5 |
| Vehicle | 41.2* | 3.34 | 0.000 | 8 |
| VPC1, 2.5M cells injected at Day 1 | 34.1 | 3.55 | 0.166 | 8 |
| VPC2, 2.5M cells injected at Day 1 | 31.6** | 2.12 | 0.036 | 7 |
| VPC1, 5M cells injected at Day 1 | 39.0 | 2.82 | 0.619 | 9 |
| VPC1, 2.5M cells injected at Day 9 | 37.8 | 1.86 | 0.439 | 6 |
| VPC2, 2.5M cells injected at Day 9 | 37.5 | 2.78 | 0.406 | 10 |
| VPC1, 5M cells injected at Day 9 | 38.5 | 2.10 | 0.481 | 10 |

The increase in the pulmonary pressures caused a rise in the right-ventricle afterload, which led to right ventricular (RV) hypertrophy, as directly shown by the Fulton's index (right ventricle vs. left ventricle ratio) which is 2.7 time higher in the SuHx vehicle group than in the normoxic healthy group (Group 1) (Table 10). PAH is characterized by a short-term right ventricular hypertrophy during which myocardial thickness increases significantly, followed by a long-term distension of the right ventricle, along with fibrosis of the right ventricle. Within the study duration of 21 days, the rat model is generally not long enough to observe significant right ventricular distension. In this study, the increase in Fulton's index clearly indicate significant hypertrophy of the right ventricle. These data also indicate that there was no effect in the development of right heart hypertrophy by cell injections.

TABLE 10
Effect of VPC1 and VPC2 on Fulton's
index of sugen-hypoxia induced PAH rat

| | Fulton's | | | |
|---|---|---|---|---|
| Treatment | Index | SEM | p value | n= |
| Normoxic Control | 0.219 | 0.06 | n/a | 5 |
| Vehicle | 0.586* | 0.05 | 0.000 | 8 |
| VPC1, 2.5M cells injected at Day 1 | 0.602 | 0.04 | 0.470 | 10 |
| VPC2, 2.5M cells injected at Day 1 | 0.608 | 0.03 | 0.373 | 10 |
| VPC1, 5M cells injected at Day 1 | 0.568 | 0.03 | 0.849 | 9 |
| VPC1, 2.5M cells injected at Day 9 | 0.568 | 0.03 | 0.858 | 8 |
| VPC2, 2.5M cells injected at Day 9 | 0.585 | 0.03 | 0.630 | 10 |
| VPC1, 5M cells injected at Day 9 | 0.657 | 0.02 | 0.072 | 10 |

The pulse pressure is considered normal when it is higher than 25% of the systolic pressure. For the normal group, the pulse pressure is 26% of the systolic pressure. (Table 11). For the SuHx+vehicle animals, the pulse pressure fell to 22% of the systolic pressure. Sugen hypoxia-induced PAH is not considered to affect myocardial inotropy; however, poor gas exchanges due to PAH cause a biphasic hypoxic effect on the left-ventricle, which eventually becomes chronically hypoxic and loses contractility strength.

TABLE 11
Effect of VPC1 and VPC2 on the pulse pressure
of sugen-hypoxia induced PAH rat

| | Pulse | | | |
|---|---|---|---|---|
| | Pressure | | | |
| Treatment | (mmHg) | SEM | p value | n= |
| Normoxic Control | 38.5 | 1.50 | n/a | 4 |
| Vehicle | 25.4* | 2.71 | 0.008 | 7 |
| VPC1, 2.5M cells injected at Day 1 | 28.4 | 3.16 | 0.498 | 8 |
| VPC2, 2.5M cells injected at Day 1 | 29.7 | 1.43 | 0.215 | 6 |
| VPC1, 5M cells injected at Day 1 | 24.3 | 2.10 | 0.750 | 9 |
| VPC1, 2.5M cells injected at Day 9 | 25.3 | 4.42 | 0.978 | 7 |
| VPC2, 2.5M cells injected at Day 9 | 28.3 | 1.68 | 0.357 | 9 |
| VPC1, 5M cells injected at Day 9 | 27.2 | 1.98 | 0.596 | 9 |

The oxygen saturation (SO₂), is considered normal between 95 and 100%. In the Control group the SO₂ was 98.6%; it fell to 88.4% in the vehicle group (Table 12), confirming that the hypertension which set in the lungs had an effect on systemic oxygenation.

TABLE 12
Effect of VPC1 and VPC2 on SO2 of
sugen-hypoxia induced PAH rat

| | SO2 | | | |
|---|---|---|---|---|
| Treatment | (%) | SEM | p value | n= |
| Normoxic Control | 98.6 | 0.75 | n/a | 5 |
| Vehicle | 88.4* | 2.09 | 0.002 | 5 |
| VPC1, 2.5M cells injected at Day 1 | 93.0 | 3.00 | 0.284 | 2 |
| VPC2, 2.5M cells injected at Day 1 | 95.7** | 0.33 | 0.041 | 3 |
| VPC1, 5M cells injected at Day 1 | 92.7 | 1.57 | 0.122 | 7 |
| VPC1, 2.5M cells injected at Day 9 | 92.3 | 3.33 | 0.340 | 4 |
| VPC2, 2.5M cells injected at Day 9 | 91.3 | 2.55 | 0.455 | 9 |
| VPC1, 5M cells injected at Day 9 | 94.6** | 0.61 | 0.008 | 9 |

Over the 21 days of the study, the normal healthy rats gained 68 g while the SuHx-vehicle animals gained an average of 21 g (Table 13). With the slower increase in body weight should come a relatively smaller gain in organ weight; however, remodeling and inflammation/oedema contribute to enhanced organ weight, and measuring lung weight is therefore a basic, but rapid, means of estimating inflammation/oedema as well as remodeling. The lungs of the vehicle treated rats were 1.8 fold heavier than in the normal rats (Table 14). The marked increased in lung weight suggest important lung oedema, embolism, or fibrosis, all of which are also characteristics of PAH. SuHx-induced PAH is characterized by an initial vasoconstriction of the pulmonary vasculature, to which some of the pulmonary gain in weight can be attributed (vascular smooth muscle hypertrophy).

TABLE 13
Effect of VPC1 and VPC2 on weight
gain of sugen-hypoxia induced PAH

| | Weight Gain | | | |
|---|---|---|---|---|
| Treatment | (g) | SEM | p value | n= |
| Normoxic Control | 68.0 | 19.53 | n/a | 5 |
| Vehicle | 20.6* | 6.47 | 0.019 | 8 |
| VPC1, 2.5M cells injected at Day 1 | 25.1 | 4.42 | 0.442 | 10 |
| VPC2, 2.5M cells injected at Day 1 | 15.4 | 8.94 | 0.720 | 10 |
| VPC1, 5M cells injected at Day 1 | 26.7 | 5.84 | 0.389 | 9 |
| VPC1, 2.5M cells injected at Day 9 | 17.6 | 4.39 | 0.831 | 8 |
| VPC2, 2.5M cells injected at Day 9 | 23.1 | 4.26 | 0.608 | 10 |
| VPC1, 5M cells injected at Day 9 | 10.6 | 4.73 | 0.265 | 10 |

TABLE 14
Effect of VPC1 and VPC2 on lung weight
of sugen-hypoxia induced PAH

| | Relative | | | |
|---|---|---|---|---|
| | Lung | | | |
| | Weight | | | |
| Treatment | (%) | SEM | p value | n= |
| Normoxic Control | 0.589 | 0.02 | n/a | 5 |
| Vehicle | 1.063* | 0.07 | 0.000 | 8 |
| VPC1, 2.5M cells injected at Day 1 | 1.095 | 0.08 | 0.901 | 10 |
| VPC2, 2.5M cells injected at Day 1 | 1.227 | 0.08 | 0.179 | 10 |
| VPC1, 5M cells injected at Day 1 | 1.128 | 0.07 | 0.615 | 9 |
| VPC1, 2.5M cells injected at Day 9 | 1.130 | 0.08 | 0.647 | 8 |
| VPC2, 2.5M cells injected at Day 9 | 0.967 | 0.06 | 0.196 | 10 |
| VPC1, 5M cells injected at Day 9 | 1.270 | 0.07 | 0.054 | 10 |

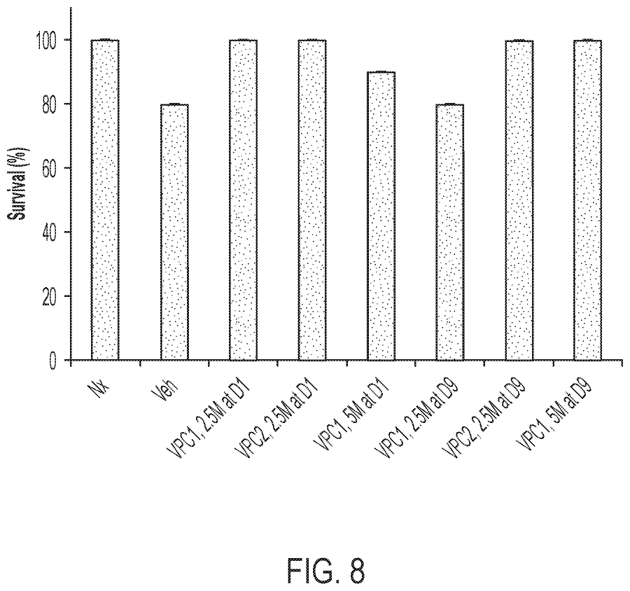

The survival rate of the SuHx+vehicle was measured at 80%; 2 out of 10 animals died before the surgery day (FIG. 8). This is compatible with internal historical mortality rates for RNU rats in this model.

VPC1. VPC1 was tested at 2 different doses; 2.5 millions of cells and 5 millions of cells. Each dose was injected to one group of animals on Day 1 (group 3 and 5 respectively) and one group on Day 9 (group 6 and 8 respectively). None of the doses tested caused a statistically significant change in the pulmonary pressures (systolic, diastolic and mean) when compared to the SuHx non-treated group (Tables 7, 8, and 9). Consequently, none of the VPC1 doses significantly prevented the increase in the Fulton's index (Table 10), suggesting that VPC1 may not prevent the right ventricular (RV) hypertrophy associated with the PAH.

The pulse pressure, mean arterial pressures and heart rate were unchanged by VPC1 treatment when compared to the vehicle group (Tables 11, 9, and 15).

TABLE 15
Effect of VPC1 and VPC2 on heart rate
of sugen-hypoxia induced PAH rat

| | Heart Rate | | | |
|---|---|---|---|---|
| Treatment | (bpm) | SEM | p value | n= |
| Normoxic Control | 376.0 | 14.97 | n/a | 4 |
| Vehicle | 299.4* | 14.30 | 0.007 | 7 |
| VPC1, 2.5M cells injected at Day 1 | 326.4 | 13.03 | 0.186 | 8 |
| VPC2, 2.5M cells injected at Day 1 | 325.8 | 22.92 | 0.335 | 6 |
| VPC1, 5M cells injected at Day 1 | 317.8 | 9.90 | 0.294 | 9 |
| VPC1, 2.5M cells injected at Day 9 | 334.7 | 16.37 | 0.132 | 6 |
| VPC2, 2.5M cells injected at Day 9 | 296.4 | 13.89 | 0.884 | 10 |
| VPC1, 5M cells injected at Day 9 | 311.6 | 12.30 | 0.531 | 10 |

The SO₂ in the Negative Control SuHx group was 88%, a value below the normal saturation range (95 to 100%) (Table 12). The SO₂ in the group treated with VPC1 at 2.5M and 5M cells at Day 1 was 93% and 92%, respectively, a little higher than the negative control group (Table 12). In the group treated with VPC1 at 5M cells on Day 9, the SO₂ was 95% (Table 12), which is within the range considered normal and healthy animals.

Relative lung weight was not modified in the VPC1 treated group compared to the vehicle group (Table 14), suggesting that VPC1 may not prevent lung fibrosis and/or associated oedema.

Over the 21 days of the study, the normal healthy rats gained 68 g while the SuHx only (vehicle group) animals gained an average of 21 g (Table 13). The animals receiving the VPC1 treatment did not gain more weight than the vehicle group (Table 13).

The survival rate in the group treated with the vehicle was 80% while it was 100% in the group treated with VPC1 at 2.5M cells at Day 1 and at 5M cells at Day 9 (FIG. 8).

The survival rate along with the general well-being and physiological parameters of the animals suggest that VPC1, at the dose of 2.5 millions cells injected either on Day 1 or 9 did not have a significant effect on SuHx-induced PAH in the rats. The dose of 5 million cells injected at Day 9 appeared to offer a small benefit, as shown by the increased oxygen saturation of the hemoglobin and the increase of the survival rate of the animals.

It should be noted that the animals did not exhibit any intolerance or adverse effects as a result of injection with VPC1. The cage side observations did not reveal any discomfort in the animals, other than the symptoms associated with the PAH.

VPC2. VPC2 was tested at the dose of 2.5 million cells. The cells were injected to one group of animals on Day 1 (group 4) and one group on Day 9 (group 7).

The systolic, diastolic, and mean pulmonary pressures in the group treated with VPC2 at Day 1 were statistically lower (by 22%, 24%, and 23%, respectively) when compared to the vehicle animals (Tables 7, 8, and 9). This suggest that VPC2, at 2.5 million cells injected at Day 1, allowed a better blood flow through the pulmonary arteries by either preventing the remodeling of the tissues and/or preventing the vasoconstriction of the pulmonary arteries caused by the sugen-hypoxia and its damage of the endothelial cells.

However, the Fulton's index increased (Table 10), suggesting that the effect of VPC2 on the hemodynamics of the animals was insufficient to prevent the right ventricular (RV) hypertrophy associated with the PAH. Furthermore, the pulse pressure, mean arterial pressure and heart rate were not statistically different in the groups treated by VPC2 (at Day 1 or Day 9) compared to the group treated with the vehicle only (Tables 11, 9, and 15).

The SO₂ in the Negative Control SuHx group was 88%, a value below the normal saturation range (95 to 100%). The SO₂ in the group treated with VPC2 at 2.5M at Day 1 was back to normal value range, a statistically and clinically significant benefit (Table 12).

Relative lung weight was not statistically significant in the group treated with VPC2 compared to vehicle group (Table 14).

The weight gain of the animals receiving VPC2 was not different from that of the animals receiving the vehicle (Table 13). The survival rate of the group treated with vehicle only was 80% while in the group treated with VPC2 at 2.5 millions cells at Day 1 or Day 9 was 100% (FIG. 8), suggesting that VPC2 protected to some extent the animals suffering from PAH.

The decrease of the pulmonary pressures, the better saturation along with the greater survival rate of the animals suggest that VPC2 offers some benefit in SuHx-induced PAH in rats.

Discussion

This pulmonary arterial hypertension study involved RNU rats, which have been found to develop a very severe and rapid form of PAH in these experimental conditions. The final experimental conditions used in this study were found to cause severe pulmonary hemodynamics impairment in the animals while maintaining mortality below 20% over 21 days.

The rapidity of the progression of the disease, and the severity of the symptoms after as little as 3 weeks represented a concern; with a disease progressing so fast, producing any therapeutic benefit to the animals represented a significant challenge. While it is conceivable that a powerful vasodilator could have prevented the onset of the disease and its early progression, the mechanism of action of the test articles in this study was not favored by such a rapid study.

Despite this, the injection of 2.5 million VPC 2 cells on Day 1 lowered the systolic, diastolic, and mean pulmonary pressure, the latter from 41.2 mmHg to 31.6 mmHg, a statistically significant benefit, and more importantly, getting the animals' mean pulmonary pressure back into a range where normal physical activity remains a possibility (25 to 35 mmHg). Combined with the increase in oxygen saturation, this suggests that 2.5 million VPC 2 cells administered on Day 1 can significantly improve pulmonary hemodynamics and remove the sustained hypoxia which lead to chronic ischemia and lung remodeling in clinical PAH patients.

Furthermore, examination of the functional endpoints of the study reveals differences between VPC 1 and VPC 2: in all cases, VPC 2 cells injected at a density of 2.5 million on Day 1 produced results which were superior to an injection of 2.5 million VPC 1 cells on the same day. This was surprising since HBs were previously shown to have an effect in a murine hind limb ischemia model and in a murine myocardial infarct model. See U.S. Pat. No. 9,938,500. Furthermore, injecting 2.5 million VPC 2 cells on Day 1 produced better results than injecting 2.5 million VPC 2 cells on Day 9, when considering the pulmonary hemodynamics and all other functional parameters measured.

Altogether, this study demonstrated the efficacy of VPC2 (HEs) cells in an extremely aggressive and rapid induced PAH syndrome involving RNU rats. While there are some reports associating a greater severity of PAH in immunodeficient patients, a progression as rapid and severe as the PAH induced in this study is unheard of in the clinic. Provided with more time and a less extreme pulmonary arterial hypertension, it is expected that the functional benefits associated with a single IV injection of VPC 2 cells (HEs) would be more favorable than suggested by the current data set.

### Example 8: Histopathological Analysis

Pulmonary arterial hypertension (PAH) is characterised by a marked and sustained elevation of pulmonary arterial pressure. The chronic alveolar hypoxia, due to lung disease or to other causes of reduced oxygen availability in animal models, leads to a sustained increase in pulmonary vascular resistance and pulmonary hypertension. Multiple factors are involved in the pathobiology of PAH, in which sustained vasoconstriction and remodelling of the pulmonary vessel wall appears to be most important. While vasoconstriction is a reversible reaction of the smooth muscle cells to a variety of stimuli, it is necessary in sustaining remodelling, which occurs in all layers of the vessel wall, and eventually leads to a more permanent restriction of the luminal diameter.

In this study, various parameters were analyzed in the animals tested in Example 7 to determine whether the hemogenic endothelial cells tested interfered with the development of the structural lesions that characterize the pulmonary vascular changes in the PAH model.

Materials and Methods

The left lobes of the lungs harvested from every rat in every experimental group (shown in Table 6) were perfused and fixed with 10% formalin before being sent to the IRIC (The Institute for Research in Immunology and Cancer in Montreal, Quebec, Canada) to make slides for the histopathological analysis.

A transversal section of the middle left lobe was cut and embedded in paraffin, sliced at 5 μm thickness, mounted and stained with Hematoxylin and Eosin (H&E).

Each slice was visualized at a 200× magnification on a Nikon Eclipse T100 microscope. A minimum of 10 non-overlapping viewfields per lung were randomly selected. Microphotographs were taken using a Nikon DS-Fi1 digital camera using Nikon NIS Elements 4.30. The photographer was blind to the treatment given the rats and features of interest. For the 10 viewfields, a single well-focused microphotograph of each area was taken and saved. All vessels found in each viewfield were analyzed, from the largest to the smallest, with no threshold or limit in vessel size.

Intra-acinar vessels i.e vessels within gas exchange regions of the lung, associated with alveoli, alveolar ducts and respiratory bronchioles were identified. All vessels associated with terminal bronchioles and all larger airways were excluded.

Vessels were divided into three size groups based on lumen diameter; small, (10-50 microns), medium (50-100 microns) or large (>100 microns) by measuring the longest axis of transected lumen. Diameters were measured using “Infinity Analyze 5.0.3.” at the widest point of the lumen, measured perpendicular to the long axis of the vessel. The lumen lied between the inner edges of the inner elastic lamina i.e. the inner elastic lamina did not form part of lumen but was considered a part of the vessel wall.

Each vessel was also categorized as non-muscular, semi-muscular or muscular.

Completely muscular. Surrounded completely (>90% circumference) by a smooth muscle layer as identified by staining and by inner and outer elastic laminae. In muscularized vessels, the external diameter was measured at the same point as the internal diameter was measured in non-muscular vessels, and ran from the outer edge to the opposite outer edge of the external elastic lamina.

Partially muscular: incompletely surrounded (10-90% circumference) by a crescent of smooth muscle and two elastic laminae for part of the circumference. In partially muscularized vessels, the external diameter was measured at the same point as the internal diameter was measured in non-muscular vessels, and runs from the outer edge to the opposite outer edge of the outermost elastic lamina at that point (whether this is the internal or external elastic lamina).

Non-muscular: a single elastic lamina for all of the circumference (<10%) of the vessel with no apparent smooth muscle layer.

Analysis

Values are presented as means±SEM (standard error of the means). Repeat unpaired Student's t-tests were performed on all experimental conditions, comparing the following groups:

SuHx group (Negative control) animals were compared to healthy animals (Normoxic Control) to confirm the successful induction of the disease. Treatment groups with the negative control animals (SuHx). Differences were considered significant when p 0.05.

Throughout, * indicates that the value is significantly different from the control (no SuHx) group while ** indicates that the value is significantly different from the negative control (SuHx) group.

Results

Effect of Sugen

Injection of Sugen caused combinations of small pulmonary medial and adventitial thickening and severe arteriopathy, including concentric neointimal and complex plexiform-like lesions. There are two patterns of complex lesion formation observed: one with the lesion forming within the vessel lumen, and another that projected outside the vessel (aneurysm-like). A third structural consequence of Sugen-induction of PAH developed much later in the progression of the disease, and consisted in the appearance of fibrosis within the pulmonary parenchyma. The preclinical Sugen-induced PAH is not a fibrotic model per say, but close examination of late-stage embedded and stained tissues allows a reliable qualification of fibrosis. The appearance of fibrosis is indicative of irreversible PAH, such as is observed in long-suffering patients. Sadly, these patients tend to be unresponsive to the current crop of vasodilator therapies for PAH.

The thickness of the walls of the small pulmonary arteries and arterioles, categorization of vessels, the population of proliferative cells (progenitor cells) surrounding these arteries, and the relative diameter of the lumen of the arteries were selected to determine the severity of the morphometric changes observable between healthy and PAH lungs. Infiltration of mononuclear inflammatory cells (alveolar macrophages) and leucocytes (lymphocyte-like cells and clusters of eosinophils) in lungs, interstitial/alveolar oedema and fibrosis in the lungs, as well as plexiform-like lesions, were also used as indices of the lung's pathophysiological state.

The severity of the histopathological changes, such as thickening of the medial arteries, infiltration of “progenitor” cells in the adventitia of small arteries and infiltration of alveolar macrophages in lung parenchyma, alveolar oedema and fibrosis and plexiform-like-lesions formation was scored from 0 to 3 where 0=none, 1=mild, 2=moderate, and 3=severe.

Arterial size, luminal diameter, presence or absence of muscularization of the arterioles were compiled from the lungs of SuHx-induced PAH rats treated with VPC1 and VPC2 as well as negative control animals shown in Table 6.

Negative Control Rats

As expected, lung tissues of control (Normoxic) animals were mainly constituted of nonmuscular arterioles (88.3%) (Tables 16, 17, and 18). In contrast, lung tissues in the negative control (SuHx) animals were mainly constituted of muscular arterioles (83.9%) (Tables 16, 17, and 18). This observation is consistent with the hyperplasia observed in the 56 days Sugen-Hypoxia model in Sprague-Dawley rats. The 11 days of hypoxia at 17% oxygen following Sugen injection, were sufficient to cause a constant pulmonary vascular smooth muscle (VSM) constriction that leads to VSM hypertrophy and hyperplasia, with the multiplication of VSM cells turning normally non-muscular arterioles into partially or fully muscularized arterioles. This increases wall thickness and decreases luminal space in those vessels. In addition, the following 10 days in a normoxic environment nonetheless maintain hypoxic conditions within the lungs due to the pulmonary smooth muscle remodeling. The hypoxic phase of the study is characterized by a rapid endothelial proliferation, which gives rise to plexiform lesions of various grades. At the end of 21 days, those lesions were often large enough to obliterate small-diameter arterioles altogether.

TABLE 16
Effect of VPC1 and VPC2 on percentage of non-
muscular vessels of SuHx-induced PAH rat

| | Non | | | |
|---|---|---|---|---|
| | muscular | | | |
| | Vessels | | | |
| Treatment | (%) | SEM | p value | n= |
| Normoxic Control | 88.32 | 1.99 | n/a | 5 |
| Vehicle | 7.45* | 1.07 | 0.000 | 10 |
| VPC1, 2.5M cells injected at Day 1 | 25.45** | 5.36 | 0.004 | 10 |
| VPC2, 2.5M cells injected at Day 1 | 46.43** | 4.88 | 0.000 | 10 |
| VPC1, 5M cells injected at Day 1 | 20.21** | 5.48 | 0.028 | 9 |
| VPC1, 2.5M cells injected at Day 9 | 14.52** | 2.62 | 0.016 | 8 |
| VPC2, 2.5M cells injected at Day 9 | 14.75** | 2.01 | 0.005 | 10 |
| VPC1, 5M cells injected at Day 9 | 14.94** | 1.99 | 0.004 | 10 |

TABLE 17
Effect of VPC1 and VPC2 on percentage of muscular vessels of
SuHx-induced PAH rat

| | Muscular | | | |
|---|---|---|---|---|
| | Vessels | | | |
| Treatment | (%) | SEM | p value | n= |
| Normoxic Control | 4.78 | 1.58 | n/a | 5 |
| Vehicle | 83.93* | 2.55 | 0.000 | 10 |
| VPC1, 2.5M cells injected at Day 1 | 63.73** | 5.09 | 0.002 | 10 |
| VPC2, 2.5M cells injected at Day 1 | 44.99** | 5.11 | 0.000 | 10 |
| VPC1, 5M cells injected at Day 1 | 69.00** | 6.38 | 0.037 | 9 |
| VPC1, 2.5M cells injected at Day 9 | 78.38 | 3.37 | 0.199 | 8 |
| VPC2, 2.5M cells injected at Day 9 | 77.14 | 2.08 | 0.054 | 10 |
| VPC1, 5M cells injected at Day 9 | 77.26 | 2.53 | 0.080 | 10 |

TABLE 18
Effect of VPC1 and VPC2 on percentage of semi-
muscular vessels of SuHx-induced PAH rat

| | Semi- | | | |
|---|---|---|---|---|
| | muscular | | | |
| | Vessels | | | |
| Treatment | (%) | SEM | p value | n= |
| Normoxic Control | 6.91 | 1.29 | 1.29 | 5 |
| Vehicle | 8.62 | 1.85 | 1.85 | 10 |
| VPC1, 2.5M cells injected at Day 1 | 10.82 | 1.44 | 1.44 | 10 |
| VPC2, 2.5M cells injected at Day 1 | 8.57 | 1.86 | 1.86 | 10 |
| VPC1, 5M cells injected at Day 1 | 10.78 | 1.54 | 1.54 | 9 |
| VPC1, 2.5M cells injected at Day 9 | 7.10 | 1.23 | 1.23 | 8 |
| VPC2, 2.5M cells injected at Day 9 | 8.11 | 0.58 | 0.58 | 10 |
| VPC1, 5M cells injected at Day 9 | 7.80 | 0.78 | 0.78 | 10 |

In the control (Normoxic) group, most of the vessels 88%) were characterized as “small” size (less than 50 microns in diameter) and were mainly nonmuscular (Tables 16, 17, and 18). Nearly 12% of vessels were described as “medium” size, while the remaining very few vessels were considered “large”. PAH induction by SuHx alters the thickness of the vessels, leading to a shift in distribution of vessels based on size, 60%—characterized as small, 38% as medium and the remaining as large vessels at the end of the study). The changes induced by SuHx were evident in the thickening of the muscle layer within the blood vessels (as shown in Tables 19, 20, and 21); small-size and medium-size pulmonary blood vessels significantly increased their musculature by 16 to 42% and 20 to 33% respectively as compared to control (Normoxic) animals. The wall thickness of large vessels did not change significantly.

TABLE 19
Effect of VPC1 and VPC2 on small vessels
wall thickness of SuHx-induced PAH rat

| | Small | | | |
|---|---|---|---|---|
| | Vessels - | | | |
| | Wall | | | |
| | Thickness | | | |
| Treatment | (%) | SEM | p value | n= |
| Normoxic Control | 16.35 | 0.97 | n/a | 5 |
| Vehicle | 41.92* | 1.98 | 0.000 | 10 |
| VPC1, 2.5M cells injected at Day 1 | 31.78** | 2.36 | 0.004 | 10 |
| VPC2, 2.5M cells injected at Day 1 | 25.68** | 1.73 | 0.000 | 10 |
| VPC1, 5M cells injected at Day 1 | 34.81** | 2.69 | 0.045 | 9 |
| VPC1, 2.5M cells injected at Day 9 | 39.16 | 1.98 | 0.344 | 8 |
| VPC2, 2.5M cells injected at Day 9 | 40.03 | 1.33 | 0.439 | 10 |
| VPC1, 5M cells injected at Day 9 | 38.70 | 1.45 | 0.206 | 10 |

TABLE 20
Effect of VPC1 and VPC2 on medium vessels
wall thickness of SuHx-induced PAH rat

| | Medium | | | |
|---|---|---|---|---|
| | Vessels - | | | |
| | Wall | | | |
| | Thickness | | | |
| Treatment | (%) | SEM | p value | n= |
| Normoxic Control | 20.06 | 0.96 | n/a | 5 |
| Vehicle | 33.18* | 1.08 | 0.000 | 10 |
| VPC1, 2.5M cells injected at Day 1 | 32.24 | 1.35 | 0.594 | 10 |
| VPC2, 2.5M cells injected at Day 1 | 28.58 | 1.96 | 0.055 | 10 |
| VPC1, 5M cells injected at Day 1 | 31.08 | 1.75 | 0.311 | 9 |
| VPC1, 2.5M cells injected at Day 9 | 32.42 | 1.75 | 0.657 | 8 |
| VPC2, 2.5M cells injected at Day 9 | 34.14 | 1.28 | 0.577 | 10 |
| VPC1, 5M cells injected at Day 9 | 34.97 | 0.94 | 0.228 | 10 |

TABLE 21
Effect of VPC1 and VPC2 on large vessels
wall thickness of SuHx-induced PAH rat

| | Large | | | |
|---|---|---|---|---|
| | Vessels - | | | |
| | Wall | | | |
| | Thickness | | | |
| Treatment | (%) | SEM | p value | n= |
| Normoxic Control | n/a | n/a | n/a | 5 |
| Vehicle | 23.42 | 2.10 | n/a | 10 |
| VPC1, 2.5M cells injected at Day 1 | 17.74 | 2.87 | 0.149 | 10 |
| VPC2, 2.5M cells injected at Day 1 | 19.07 | 1.67 | 0.143 | 10 |
| VPC1, 5M cells injected at Day 1 | 21.44 | 5.29 | 0.715 | 9 |
| VPC1, 2.5M cells injected at Day 9 | 22.55 | 1.58 | 0.762 | 8 |
| VPC2, 2.5M cells injected at Day 9 | 20.74 | 3.38 | 0.520 | 10 |
| VPC1, 5M cells injected at Day 9 | 25.98 | 2.13 | 0.427 | 10 |

An increase in wall thickness decreases the luminal diameter of the arteries, increasing the pulmonary arterial pressure against which the right ventricle must pump (the right-ventricular afterload).

Plexiform lesions were not observed in healthy, non-induced animals. In contrast, animals induced with Sugen but not benefiting from any treatment exhibited Grade 2 and 3 plexiform lesions, corresponding to moderate (grade 2) to severe endothelial overgrowth with some complete obliteration of the vessels lumen (grade 3). In addition to the plexiform lesions which are characteristic of human PAH, the animals not benefiting from treatment also exhibited signs of fibrosis and interstitial/alveolar edema.

VPC1

VPC1 was tested at 2 different doses; 2.5 millions of cells and 5 millions of cells. Each dose was injected to one group of animals on Day 1 and one group on Day 9.

Just as PAH induction alter the distribution of vessels based on size, treatment with VPC1 alter the distribution of vessels based on size as well. VPC1 slightly increased “small” size vessels and decreased “medium” size vessels as compared to SuHx rats only (data not shown).

The wall thickness of the small lung vessels (mostly dictated by the thickness of the smooth muscle layer), of the rats treated with VPC1 at 2.5 M and 5 M cells on day 1 was statistically lower compared to the vehicle treated rats. Wall thickness of the medium and large vessels did not change significantly (Tables 19, 20, and 21). The treatment with VPC1 on day 9 did not have any effect on vessels wall thickness.

The percentage of muscular vessels was significantly lower in VPC1-treated animals at 2.5 and 5 M cells on day 1; from 83.9% in negative control SuHx treated animals to 64% and 69% respectively in VPC1-treated animals (Tables 16, 17, and 18).

The same dose of VPC1 injected on day 9 did not have statistically significant effect on the percentage of muscular vessels in the lung tissues.

Moreover, the alveolar macrophage infiltrations, oedema/fibrosis and pulmonary artery lesions observed in the groups treated with VPC1 on day 1 were lower than in vehicle animals. The plexiform lesions in the groups treated with VPC1 on day 1 were classified as mild/moderate (score 1 to 2).

VPC2

VPC2 was tested at the dose of 2.5 million cells. The cells were injected to one group of animals on Day 1 and one group on Day 9.

Just as PAH induction alters the distribution of vessels based on size, treatment with VPC2 on day 1 alters the distribution of vessels based on size as well. VPC2 injected at day 1 increased the number of “small” size vessels and decreased “medium” size vessels as compared to SuHx rats. The treatment with VPC2 on day 1 brings the proportion of “small” size vessels versus “medium” size and “large” size vessel very close the one observed in the normoxic perfectly heathy rats.

The wall thickness in small lung vessels of rats treated with VPC2 at 2.5M cells on day 1 was statistically smaller compared to the vehicle treated rats. VPC2 administrated on day 9 did not have any effect on vessels wall thickness. Wall thickness of the medium and large vessels did not change significantly (Tables 19, 20, and 21). The treatment with VPC2 on day 9 did not have any effect on vessels wall thickness.

The percentage of muscular vessels was significantly lower in animal treated with VPC2 on day 1; from 83.9% in vehicle-treated animals to 45% in VPC2-treated animals. Consequently, the percentage of non-muscular vessels increased from 7% to 46%. VPC2 at day 9 did not have significant effect on muscular vessels. See Tables 16, 17, and 18.

These results confirm the functional findings, which showed that the symptoms of PAH in SuHx-induced animals were much less severe in animals treated with VPC2. VPC2 prevented the remodeling of the pulmonary blood vessels in the SuHx-induced PAH rat model.

Moreover, alveolar macrophage infiltrations, oedema/fibrosis and pulmonary artery lesions observed in the VPC2-treated animals were much lower than in negative control SuHx animals, classified as none/mild (score 0 to 1), suggesting that VPC2 prevents the onset of lung changes associated with PAH.

This study demonstrated the high efficacy of VPC2 (HEs) on functional as well as structural findings in an extremely aggressive and rapid induced PAH syndrome in RNU rats.

### Example 9: HE Contains a Distinct Vascular Endothelial Fraction that is VECAD+

The flow cytometry and transcript analyses above indicated that there was likely a significant vascular endothelial component generated by the HE differentiation protocol. To better define similarities and differences between the PSC-derived EC-like cells and mature ECs, we performed single cell RNA-sequencing comparing HE, HUVEC, and undifferentiated iPSCs (GMP1).

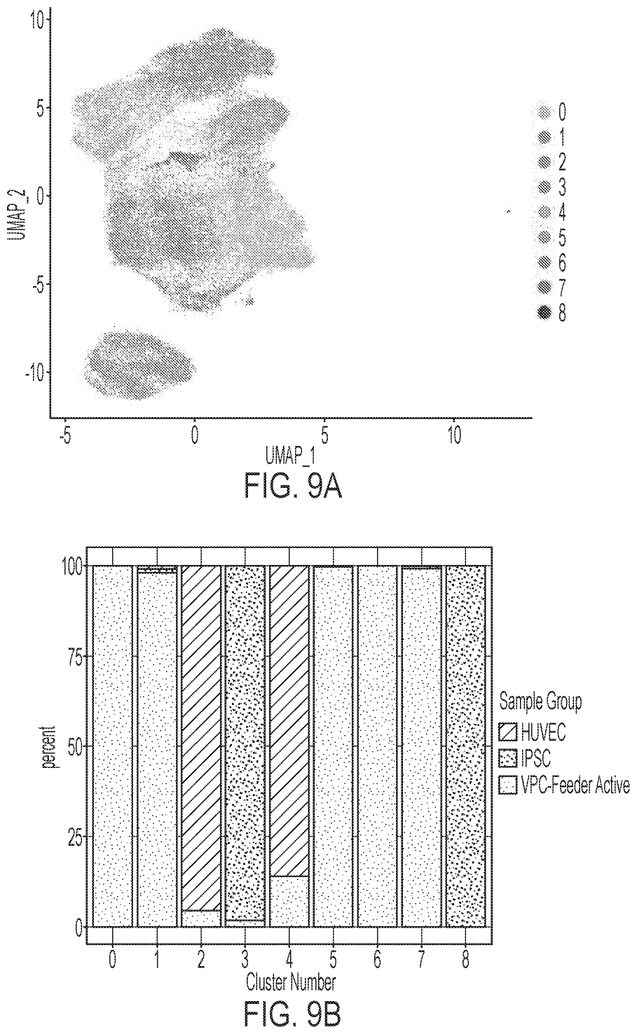

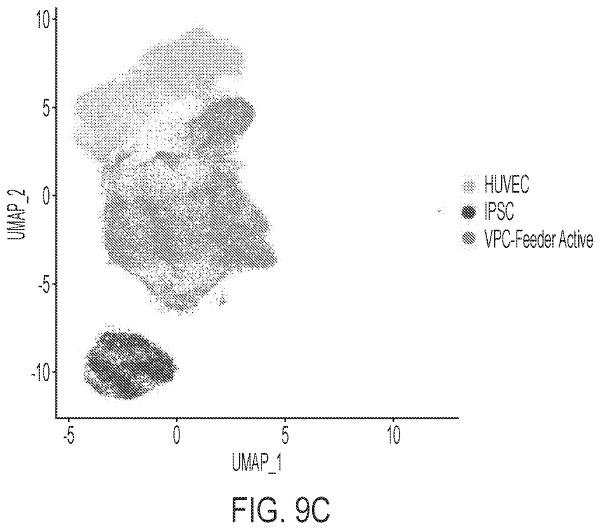

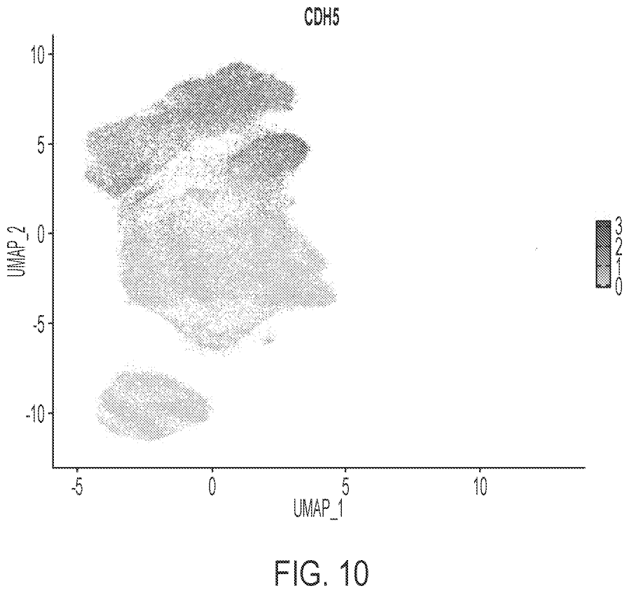

Unsupervised clustering revealed 9 clusters among the cell types tested (FIG. 9A). As expected, undifferentiated iPSCs clustered distinctly from HE cells (“VPC-feeder active”) and HUVEC (FIGS. 9B and 9C). HE were organized into multiple clusters, but overall, in a population largely separable from iPSCs and HUVECs. When the expression of specific markers of vascular endothelial cells were interrogated, three clusters were identified by the presence of VECAD/CDH5 (clusters 2, 4, and 5) (FIG. 10). Clusters 2 and 4 were composed primarily of HUVEC, while cluster 5 was composed of HE cells (FIG. 9B). Given that cluster 5 appeared to be composed of VECAD+ cells, differential gene expression analysis was conducted comparing VECAD+HE cells from cluster 5 to cells from other clusters and found that cluster 5 had a strong vascular endothelial signature, as indicated by the functions of the most differentially expressed genes (Table 22). Many of the 50 most significantly upregulated genes in cluster 5 were genes with known vascular expression and activity, and included PLVAP, GJA4, ESAM, EGFL7, KDR/VEGFR2, ESAM, and VECAD (CDH5) (Table 22). Gene ontology analysis indicated that among the most enriched pathways were EC migration, endothelium development, sprouting angiogenesis, and other EC-related processes. Similarly, gene set enrichment analysis revealed pathways important to endothelial development and function, including TGF beta signaling and hypoxia.

Clustering analysis also showed that HE cells were largely distinct from HUVECs. Cluster 5 had minimal but nonzero HUVEC contribution, and clusters 2 and 4 were composed primarily of HUVEC with small (<15%) HE representation (FIG. 9B). Differential gene expression analysis comparing cluster 5 with the clusters composed primarily of HUVEC revealed that the VECAD+HE cells in cluster 5 were immature or progenitor ECs (Table 23). Among the genes more highly expressed in the VECAD+HE cells were SOX9, PDGFRA, and EGFRA, which are markers of replicative vessel-borne progenitor vascular cells that are antecedents to terminally differentiated ECs. A recent study (Kutikhin, A. G. et al. Cells 9:876 (2020)) comparing endothelial colony-forming cells (ECFCs) with mature vessel-borne endothelial cells (ECs) identified KDR/VEGFR2, NOTCH4, and collagen I and IV subunits as ECFC-enriched factors, and those transcripts were similarly upregulated in the VECAD+HE cells of cluster 5 compared to HUVEC, although other ECFC-enriched genes such as CD34 were not higher in the HE cells. While HE cells and HUVEC expressed VECAD/CDH5 and PECAM1/CD31, HUVEC levels were higher, which again is consistent with HE cells being a more immature or progenitor EC-like cell. Gene ontology analysis using the set of genes differentially expressed between VECAD+HE cells and HUVEC indicated that the most enriched pathways were sterol biosynthesis, protein kinase A signaling, digestive tract and cardiac ventricle development. When compared with iPSC, gene set enrichment analysis revealed that differentially expressed genes were associated with pathways important to endothelial development and homeostasis such as MTORC1, WNT, and TGF beta signaling. Taken together, single cell RNA sequencing revealed a cluster of HE that is similar to HUVEC, possessing qualities of a bona fide EC, but also possessing distinctive characteristics suggestive of an immature or progenitor phenotype.

TABLE 22
50 Most Significantly Upregulated Genes in Cluster
5 Compared to Cells in Other Clusters

| gene | p_val | p_val_adj | avg_logFC | pct.1 | pct.2 |
|---|---|---|---|---|---|
| GJA4 | 0.00E+00 | 0.00E+00 | 1.739765 | 0.751 | 0.171 |
| PLVAP | 0.00E+00 | 0.00E+00 | 1.710396 | 0.96 | 0.497 |
| IGFBP4 | 0.00E+00 | 0.00E+00 | 1.483074 | 0.987 | 0.675 |
| FCN3 | 0.00E+00 | 0.00E+00 | 1.425734 | 0.581 | 0.1 |
| GNG11 | 0.00E+00 | 0.00E+00 | 1.184349 | 0.973 | 0.682 |
| ESAM | 0.00E+00 | 0.00E+00 | 1.128435 | 0.898 | 0.389 |
| SLC9A3R2 | 0.00E+00 | 0.00E+00 | 1.100179 | 0.805 | 0.394 |
| CDH5 | 0.00E+00 | 0.00E+00 | 1.046682 | 0.738 | 0.164 |
| IGFBP5 | 0.00E+00 | 0.00E+00 | 1.044824 | 0.455 | 0.221 |
| SOX18 | 0.00E+00 | 0.00E+00 | 1.014948 | 0.682 | 0.161 |
| KDR | 0.00E+00 | 0.00E+00 | 0.980832 | 0.949 | 0.669 |
| GMFG | 0.00E+00 | 0.00E+00 | 0.97714 | 0.835 | 0.269 |
| HLA-E | 0.00E+00 | 0.00E+00 | 0.959084 | 0.891 | 0.491 |
| MMRN2 | 0.00E+00 | 0.00E+00 | 0.938295 | 0.666 | 0.121 |
| VAMP5 | 0.00E+00 | 0.00E+00 | 0.914077 | 0.921 | 0.612 |
| ARHGDIB | 0.00E+00 | 0.00E+00 | 0.887378 | 0.825 | 0.394 |
| ADGRL4 | 0.00E+00 | 0.00E+00 | 0.883791 | 0.703 | 0.231 |
| GJA5 | 0.00E+00 | 0.00E+00 | 0.862907 | 0.524 | 0.132 |
| EFNB2 | 0.00E+00 | 0.00E+00 | 0.862327 | 0.674 | 0.377 |
| PECAM1 | 0 | 0 | 0.846013 | 0.654 | 0.17 |
| RNASE1 | 0.00E+00 | 0.00E+00 | 0.829217 | 0.518 | 0.226 |
| ECSCR | 0.00E+00 | 0.00E+00 | 0.79933 | 0.687 | 0.176 |
| ABHD17A | 0.00E+00 | 0.00E+00 | 0.769739 | 0.854 | 0.568 |
| HSPG2 | 0.00E+00 | 0.00E+00 | 0.760038 | 0.65 | 0.323 |
| FAM107B | 0.00E+00 | 0.00E+00 | 0.758576 | 0.682 | 0.309 |
| EGFL7 | 0.00E+00 | 0.00E+00 | 0.754544 | 0.991 | 0.91 |
| MEF2C | 0.00E+00 | 0.00E+00 | 0.747243 | 0.745 | 0.343 |
| ARGLU1 | 0.00E+00 | 0.00E+00 | 0.743373 | 0.794 | 0.693 |
| FLT1 | 0.00E+00 | 0.00E+00 | 0.737392 | 0.968 | 0.891 |
| S100A16 | 0.00E+00 | 0.00E+00 | 0.728981 | 0.967 | 0.819 |
| CFLAR | 0.00E+00 | 0.00E+00 | 0.726916 | 0.783 | 0.423 |
| COTL1 | 0.00E+00 | 0.00E+00 | 0.725018 | 0.918 | 0.741 |
| SOX17 | 0.00E+00 | 0.00E+00 | 0.72092 | 0.486 | 0.153 |
| DLL4 | 0 | 0 | 0.709932 | 0.483 | 0.079 |
| PLK2 | 0.00E+00 | 0.00E+00 | 0.709154 | 0.862 | 0.584 |
| SLC2A1 | 0.00E+00 | 0.00E+00 | 0.699951 | 0.759 | 0.614 |
| ITM2B | 0.00E+00 | 0.00E+00 | 0.696128 | 0.982 | 0.942 |
| CXCR4 | 0.00E+00 | 0.00E+00 | 0.68996 | 0.513 | 0.36 |
| RAMP2 | 0.00E+00 | 0.00E+00 | 0.686508 | 0.704 | 0.488 |
| FAM69B | 0.00E+00 | 0.00E+00 | 0.681908 | 0.89 | 0.622 |
| FKBP1A | 0.00E+00 | 0.00E+00 | 0.681477 | 0.959 | 0.874 |
| PTP4A3 | 0.00E+00 | 0.00E+00 | 0.680399 | 0.65 | 0.376 |
| SERPINB6 | 0.00E+00 | 0.00E+00 | 0.676227 | 0.91 | 0.792 |
| CD9 | 0.00E+00 | 0.00E+00 | 0.673737 | 0.783 | 0.543 |
| PLXND1 | 0.00E+00 | 0.00E+00 | 0.672561 | 0.728 | 0.443 |
| CAVIN1 | 0.00E+00 | 0.00E+00 | 0.671818 | 0.779 | 0.476 |
| ENG | 0.00E+00 | 0.00E+00 | 0.671153 | 0.575 | 0.205 |
| THY1 | 0.00E+00 | 0.00E+00 | 0.6667 | 0.765 | 0.53 |
| RASIP1 | 0.00E+00 | 0.00E+00 | 0.665168 | 0.66 | 0.248 |
| HEY1 | 0.00E+00 | 0.00E+00 | 0.662962 | 0.746 | 0.45 |

TABLE 23
100 Most Significantly Upregulated Genes
in Cluster 5 Compared to HUVEC Cells

| gene | p_val | p_val_adj | avg_logFC | pct.1 | pct.2 |
|---|---|---|---|---|---|
| CRHBP | 0 | 0 | 3.333906 | 0.983 | 0.005 |
| PLVAP | 0 | 0 | 2.660275 | 0.96 | 0.105 |
| HAPLN1 | 0 | 0 | 2.582318 | 0.979 | 0.004 |
| CD24 | 0 | 0 | 2.117768 | 0.895 | 0.011 |
| FLT1 | 0 | 0 | 2.095663 | 0.968 | 0.366 |
| IGFBP2 | 0 | 0 | 2.040781 | 0.997 | 0.702 |
| CKB | 0 | 0 | 2.020425 | 0.941 | 0.027 |
| GJA4 | 0 | 0 | 1.891513 | 0.751 | 0.172 |
| SLC2A3 | 0 | 0 | 1.787711 | 0.903 | 0.118 |
| S100A4 | 0 | 0 | 1.737065 | 0.75 | 0.02 |
| KRT8 | 0 | 0 | 1.687879 | 0.973 | 0.617 |
| FCN3 | 0 | 0 | 1.686621 | 0.581 | 0.004 |
| IGFBP5 | 0 | 0 | 1.620931 | 0.455 | 0 |
| LAPTM4B | 0 | 0 | 1.544365 | 0.966 | 0.497 |
| BNIP3 | 0 | 0 | 1.531254 | 0.967 | 0.661 |
| KRT19 | 0 | 0 | 1.512069 | 0.89 | 0.271 |
| ITM2C | 0 | 0 | 1.506945 | 0.879 | 0.033 |
| SLC2A1 | 0 | 0 | 1.503243 | 0.759 | 0.119 |
| TUBB2B | 0 | 0 | 1.4743 | 0.841 | 0.005 |
| KDR | 0 | 0 | 1.443674 | 0.949 | 0.512 |
| LDHA | 0 | 0 | 1.41049 | 0.997 | 0.843 |
| APOE | 0 | 0 | 1.407249 | 0.726 | 0.029 |
| THY1 | 0 | 0 | 1.388232 | 0.765 | 0.009 |
| FAM162A | 0 | 0 | 1.381927 | 0.908 | 0.519 |
| CRABP2 | 0 | 0 | 1.351647 | 0.723 | 0.003 |
| ID1 | 0 | 0 | 1.323672 | 0.977 | 0.659 |
| COL3A1 | 0 | 0 | 1.298193 | 0.694 | 0.027 |
| NTS | 0 | 0 | 1.290051 | 0.536 | 0.002 |
| TXNIP | 0 | 0 | 1.254997 | 0.75 | 0.323 |
| QPRT | 0 | 0 | 1.25253 | 0.756 | 0.003 |
| SLC16A3 | 0 | 0 | 1.225941 | 0.91 | 0.402 |
| ENO1 | 0 | 0 | 1.207866 | 1 | 0.969 |
| TIMP3 | 0 | 0 | 1.196902 | 0.737 | 0.084 |
| GYPC | 0 | 0 | 1.193327 | 0.835 | 0.185 |
| HEY1 | 0 | 0 | 1.193009 | 0.746 | 0.095 |
| TMEM141 | 0 | 0 | 1.18403 | 0.887 | 0.621 |
| COL6A2 | 0 | 0 | 1.181263 | 0.756 | 0.026 |
| HES4 | 0 | 0 | 1.17592 | 0.805 | 0.402 |
| CD44 | 0 | 0 | 1.170072 | 0.799 | 0.217 |
| PGK1 | 0 | 0 | 1.169827 | 0.98 | 0.824 |
| BST2 | 0 | 0 | 1.162566 | 0.807 | 0.555 |
| CLEC11A | 0 | 0 | 1.16185 | 0.822 | 0.25 |
| SLC9A3R2 | 0 | 0 | 1.156233 | 0.805 | 0.533 |
| KRT18 | 0 | 0 | 1.136732 | 0.994 | 0.892 |
| FBLN1 | 0 | 0 | 1.116144 | 0.739 | 0.006 |
| PCAT14 | 0 | 0 | 1.113903 | 0.629 | 0 |
| MSMO1 | 0 | 0 | 1.108676 | 0.849 | 0.418 |
| HMGCS1 | 0 | 0 | 1.094925 | 0.791 | 0.274 |
| CXCR4 | 0 | 0 | 1.086418 | 0.513 | 0.104 |
| TPI1 | 0 | 0 | 1.079699 | 0.999 | 0.973 |
| PTP4A3 | 0 | 0 | 1.071418 | 0.65 | 0.054 |
| ITM2B | 0 | 0 | 1.05799 | 0.982 | 0.911 |
| TMEM100 | 0 | 0 | 1.05409 | 0.569 | 0.001 |
| MVD | 0 | 0 | 1.049627 | 0.797 | 0.343 |
| GJA5 | 0 | 0 | 1.03821 | 0.524 | 0.013 |
| BAMBI | 0 | 0 | 1.031801 | 0.658 | 0.043 |
| HOPX | 0 | 0 | 1.026231 | 0.681 | 0.174 |
| APOC1 | 0 | 0 | 1.024475 | 0.704 | 0.128 |
| SERPINB1 | 0 | 0 | 1.021549 | 0.799 | 0.435 |
| PGAM1 | 0 | 0 | 1.01222 | 0.986 | 0.887 |
| POMP | 0 | 0 | 1.010744 | 0.989 | 0.966 |
| TUBA1A | 0 | 0 | 1.007929 | 0.956 | 0.901 |
| ACAT2 | 0 | 0 | 1.007418 | 0.831 | 0.507 |
| BEX1 | 0 | 0 | 0.998364 | 0.615 | 0.009 |
| PRTG | 0 | 0 | 0.995446 | 0.682 | 0.108 |
| P4HA1 | 0 | 0 | 0.991764 | 0.731 | 0.207 |
| SERPINE2 | 0 | 0 | 0.977086 | 0.666 | 0.073 |
| ID3 | 0 | 0 | 0.97512 | 0.988 | 0.923 |
| CYBA | 0 | 0 | 0.970771 | 0.925 | 0.669 |
| EFNB2 | 0 | 0 | 0.964464 | 0.674 | 0.362 |
| PKM | 0 | 0 | 0.9469 | 0.995 | 0.911 |
| UNC5B | 0 | 0 | 0.927982 | 0.571 | 0.012 |
| COL4A1 | 0 | 0 | 0.925096 | 0.926 | 0.711 |
| IGDCC3 | 0 | 0 | 0.921767 | 0.578 | 0 |
| ARGLU1 | 0 | 0 | 0.915778 | 0.794 | 0.67 |
| GJA1 | 0 | 0 | 0.909177 | 0.805 | 0.616 |
| LIMD2 | 0 | 0 | 0.908494 | 0.866 | 0.562 |
| GMFG | 0 | 0 | 0.90607 | 0.835 | 0.453 |
| FDX1 | 0 | 0 | 0.904597 | 0.784 | 0.479 |
| FDFT1 | 0 | 0 | 0.895682 | 0.884 | 0.648 |
| JUND | 0 | 0 | 0.887796 | 0.908 | 0.638 |
| SERPING1 | 0 | 0 | 0.875879 | 0.592 | 0.002 |
| BEX3 | 0 | 0 | 0.875878 | 0.977 | 0.633 |
| ANGPTL4 | 0 | 0 | 0.869906 | 0.548 | 0.041 |
| PLK2 | 0 | 0 | 0.867144 | 0.862 | 0.556 |
| CA2 | 0 | 0 | 0.865318 | 0.512 | 0 |
| HLA-DRB1 | 0 | 0 | 0.85335 | 0.545 | 0 |
| PLIN2 | 0 | 0 | 0.843637 | 0.711 | 0.348 |
| COTL1 | 0 | 0 | 0.841217 | 0.918 | 0.775 |
| ABHD17A | 0 | 0 | 0.840353 | 0.854 | 0.601 |
| IGFBP4 | 0 | 0 | 0.836 | 0.987 | 0.956 |
| SERPINH1 | 0 | 0 | 0.832031 | 0.923 | 0.818 |
| C4orf3 | 0 | 0 | 0.829329 | 0.947 | 0.852 |
| IER2 | 0 | 0 | 0.826343 | 0.848 | 0.569 |
| S100A11 | 0 | 0 | 0.825409 | 0.995 | 0.992 |
| FURIN | 0 | 0 | 0.824346 | 0.724 | 0.369 |
| CSRP2 | 0 | 0 | 0.820907 | 0.665 | 0.17 |
| TIMP1 | 0 | 0 | 0.819248 | 0.973 | 0.904 |
| TCEAL9 | 0 | 0 | 0.816856 | 0.904 | 0.546 |
| FSCN1 | 0 | 0 | 0.811547 | 0.963 | 0.879 |

### Example 10: HEs Attenuate Hemodynamic Parameters and Vascular Remodeling in Rat Models of Pulmonary Arterial Hypertension

Treatment of rodents with monocrotaline (MCT) induces vascular resistance and cardiac dysfunction (Rabinovitch, M. Toxicol Pathol 19, 458-469 (1991)) and the Sugen/hypoxia model induces the aforementioned clinical markers as well as formation of plexiform lesions, a clinical hallmark of advanced disease in humans (Ciuclan, L. et al. Am J Respir Crit Care Med 184, 1171-1182 (2011)).

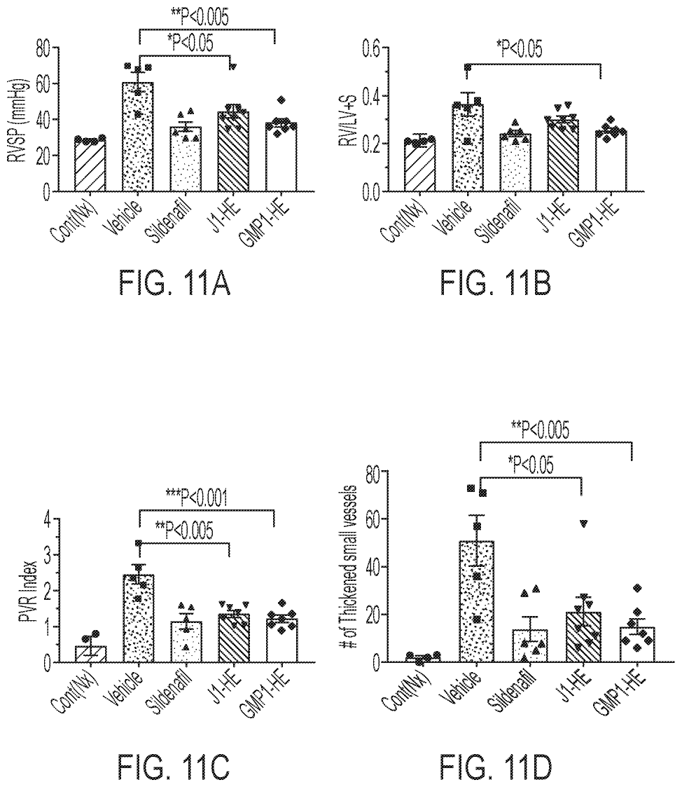

In MCT rats, treatment with HE derived from both J1-ESC and GMP-1 iPSC attenuated symptoms of PAH. Briefly, rnu/rnu rats were given a single dose of MCT (50 mg/kg, ip) at day 0. Three days later, rats were divided into vehicle, J1-HE, and GMP-1 HE groups and dosed with control medium or cells (2.5×10⁶) via intravenous injection. As a positive control, another group was given a high dose of sildenafil (˜15 mg/kg/day) in their drinking water. At day 28, hemodynamic analyses was performed by right and left heart catheterization. As expected, vehicle-treated rats showed increased right ventricle systolic pressure (RVSP), Fulton's Index, and pulmonary vascular resistance index (PVR Index) (FIGS. 11A-C). RVSP and PVR index values were lower in rats treated with J1-HE (FIGS. 11A and 11C). RVSP, Fulton's Index, and PVR index values were lower in rats treated with GMP-1-HE (FIGS. 11A-C). Histological analysis revealed that rats from the J1-HE and GMP-1-HE groups had fewer thickened vessels compared to vehicle-treated rats, which was corroborated by quantification (FIG. 11D).

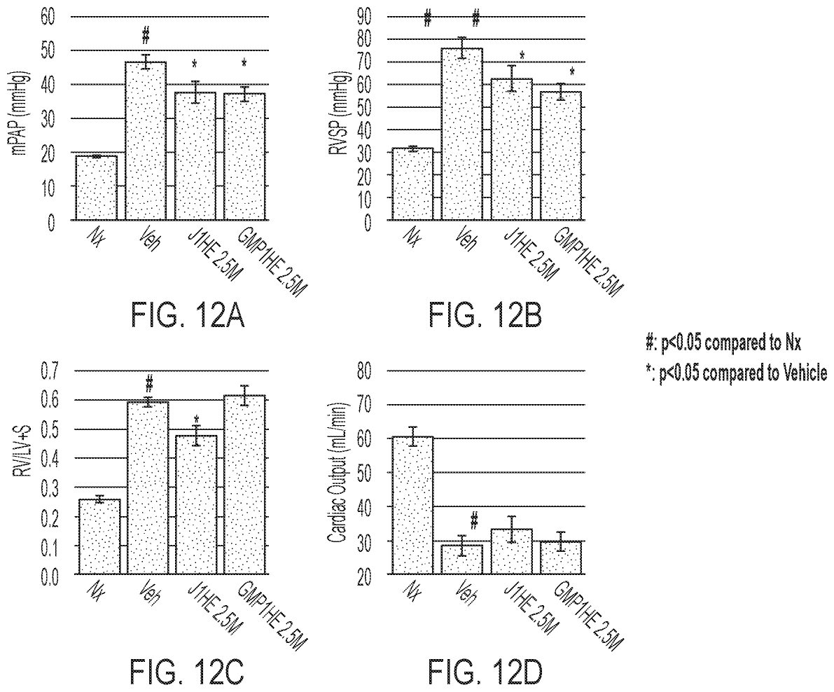

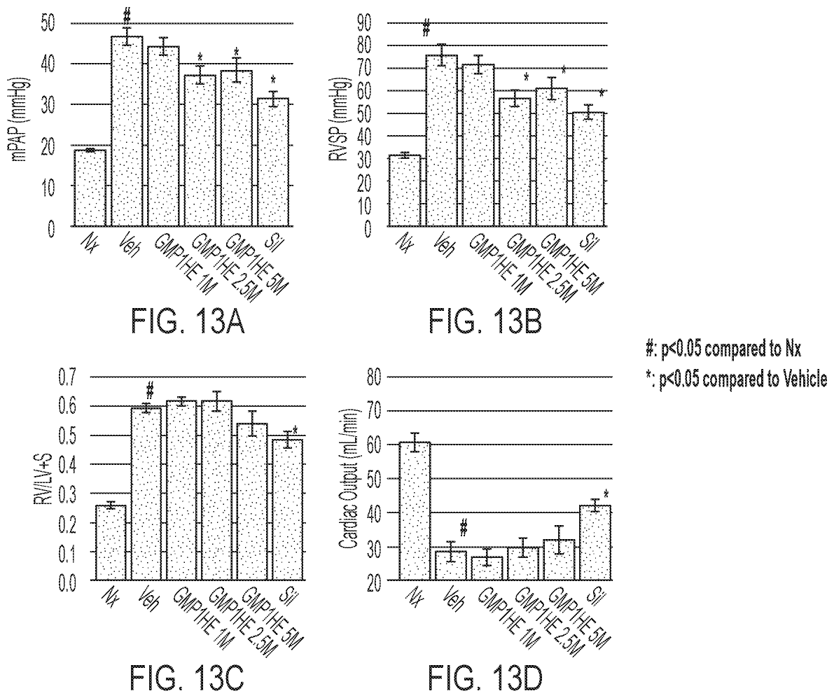

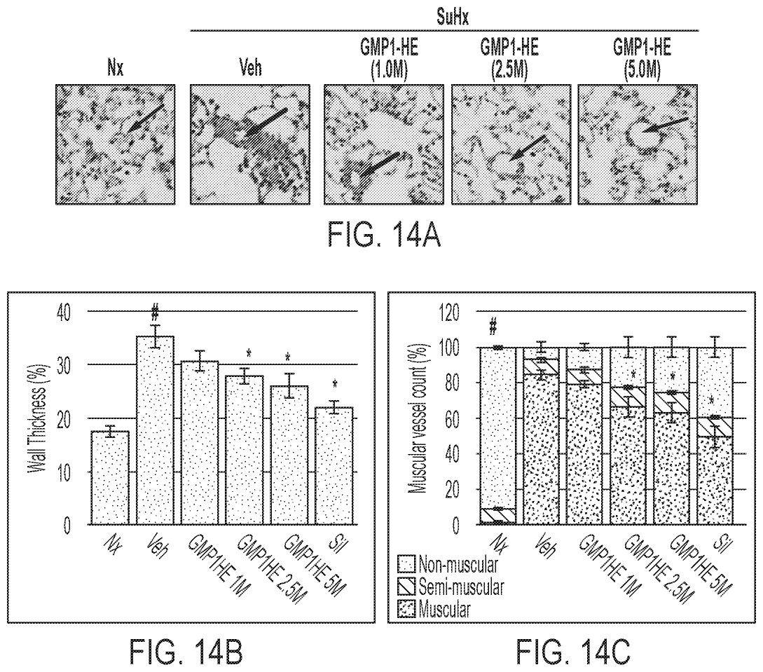

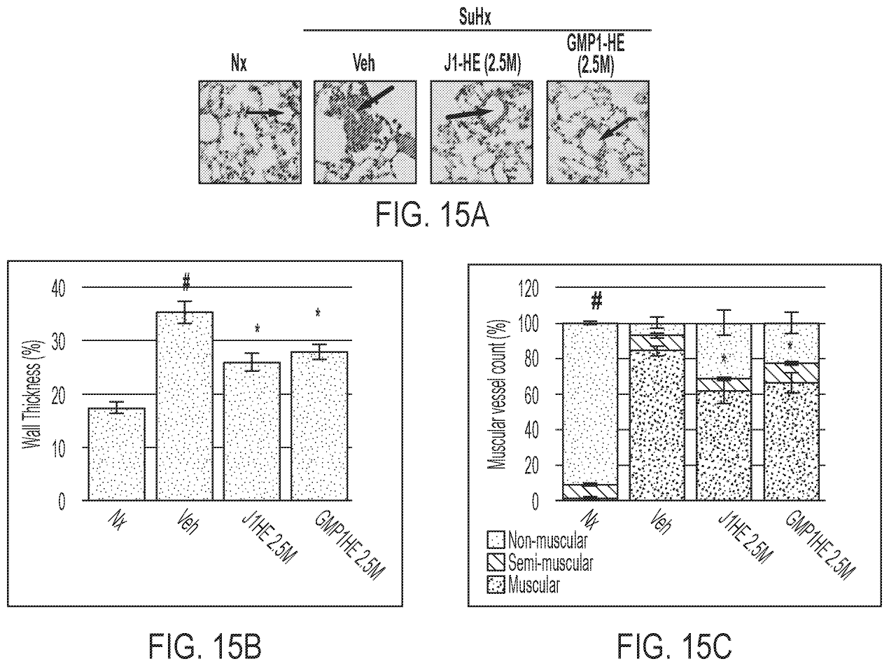

Next, the PSC-derived HEs were tested again in the Sugen/hypoxia model of PAH. In these studies, rnu/rnu rats were subjected to the sugen/hypoxia/normoxia conditions for 21 days. Rats were given a single dose of Sugen at day 0, followed by intravenous injection of vehicle, J1-HE, or GMP-1-HE at day 1 with 1 million, 2.5 million, or 5 million cells. As an additional control, another group was given sildenafil (50 mg/kg) by oral gavage twice daily. Rats treated with J1-HEs and GMP-1-HEs at 2.5 million per injection showed decreased mPAP, RVSP, and Fulton's index and improved cardiac functions such as stroke volume and cardiac output compared to vehicle-treated (FIGS. 12A-D). Furthermore, GMP-1-HE improved its efficacy in a dose dependent manner in pulmonary hemodynamics, RV remodeling, cardiac function (FIGS. 13A-D). Histological analyses of lung tissue revealed differences between control and J1-HE or GMP-1-HE-treated rats in the Sugen/hypoxia model (FIGS. 14A-C and FIGS. 15A-C). Fewer plexiform lesions could be observed in HE-treated animals compared to vehicle-treated (FIGS. 14A and 15A). Lung vessel wall thickness in HE-treated animals was also reduced compared to vehicle-treated animals (FIGS. 14B and 15B). The percentages of lung vessels categorized as muscular and semi-muscular for animals in the HE-treated groups were lower than vehicle-treated (FIGS. 14C and 15C). Lastly, HE-treated lungs had less immune cell infiltration compared to vehicle-treated animals (data not shown).

Whole transcriptome analysis of lungs from HE- and vehicle-treated rat lungs from the Sugen/hypoxia model supported the physiological data suggesting HE-treatment attenuated pathological vascular remodeling. RNA from rat lungs was collected at day 21 and differential gene expression analysis was performed. Pathway analysis of genes downregulated by ≥1.25 fold by cell treatment indicated that genes associated with smooth muscle cell development, immune cell system infiltration, and inflammation, among others, were reduced. Conversely, gene upregulated by ≥1.25 fold by cell treatment were associated with a favorable metabolic state, i.e. favoring oxidative phosphorylation, perturbation of which is associated with the PAH disease state. Taken together, these data suggest HE protect rats in models of PAH by reducing vascular resistance, vascular remodeling, and cardiac hypertrophy at a dose range of 2.5 million to 5 million per injection.

### Example 11: HEs Restore Microvasculature in the Lung

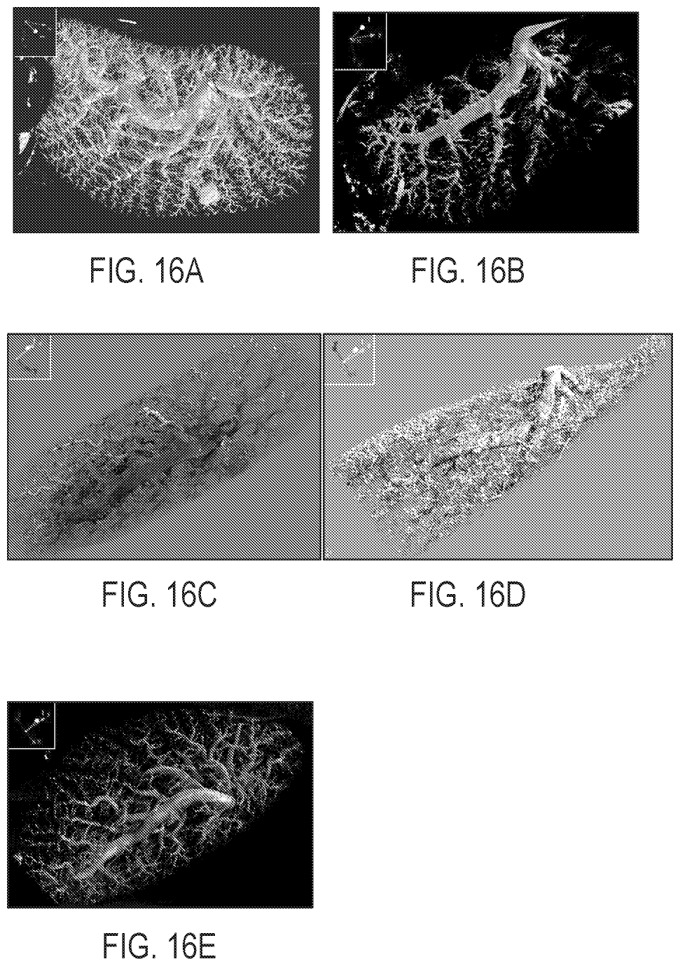

Endothelial progenitor cells are reported to preserve microvasculature in MCT treated lung (Zhao et al. Cir. Res. 96:442-450 (2005)). Therefore, micro CT scanning was performed on the lungs from the SuHx model treated with Nx control, vehicle, sildenafil, and 1 million and 5 million GMP1-HE cells. MicroCT scanning revealed an even filling of distal arteriolar bed and homogeneous pattern of capillary perfusion in normal lung (FIG. 16A). In contrast, SuHx lung treated with vehicle showed narrowed distal arteriolar bed and capillary occlusion (FIG. 16B). Treatment with 5 million HE cells (FIG. 16D) but not with 1 million HE cells (FIG. 16C) preserved microvasculature visualized by contrast agents injection. There was a marked improvement in the appearance of the lung microvasculature with preservation of arteriolar continuity and enhanced capillary perfusion with 5 million HE cells (FIG. 16D. Treatment of sildenafil showed modest improvement on capillary perfusion (FIG. 16E).

### Example 12: HEs Contain a Distinct Vascular Endothelial Fraction that is Therapeutically Active

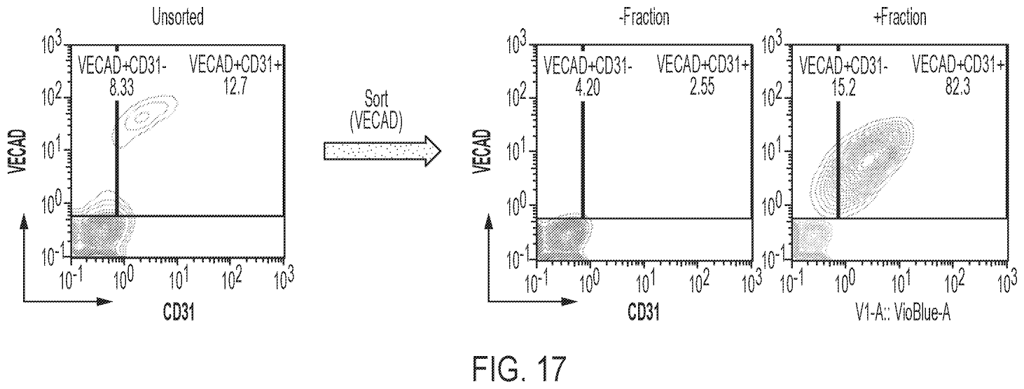

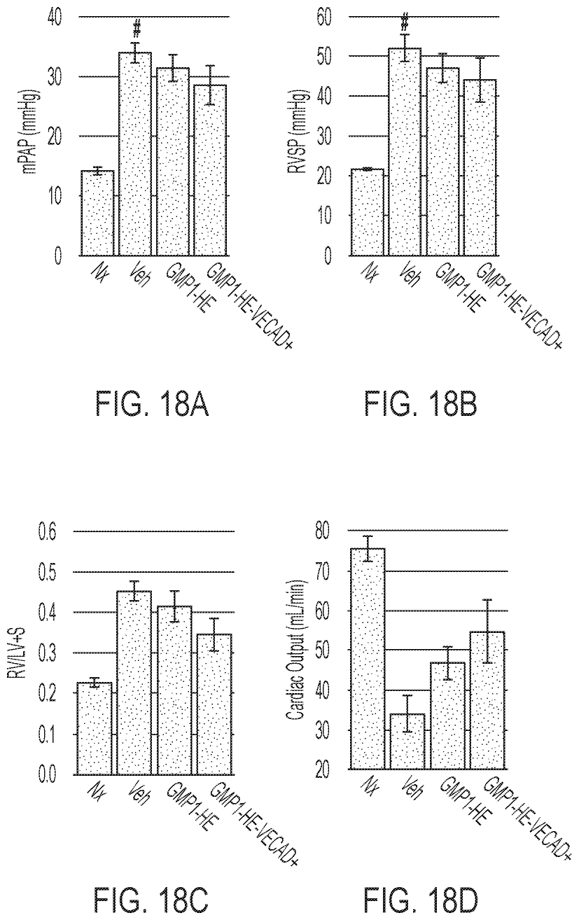

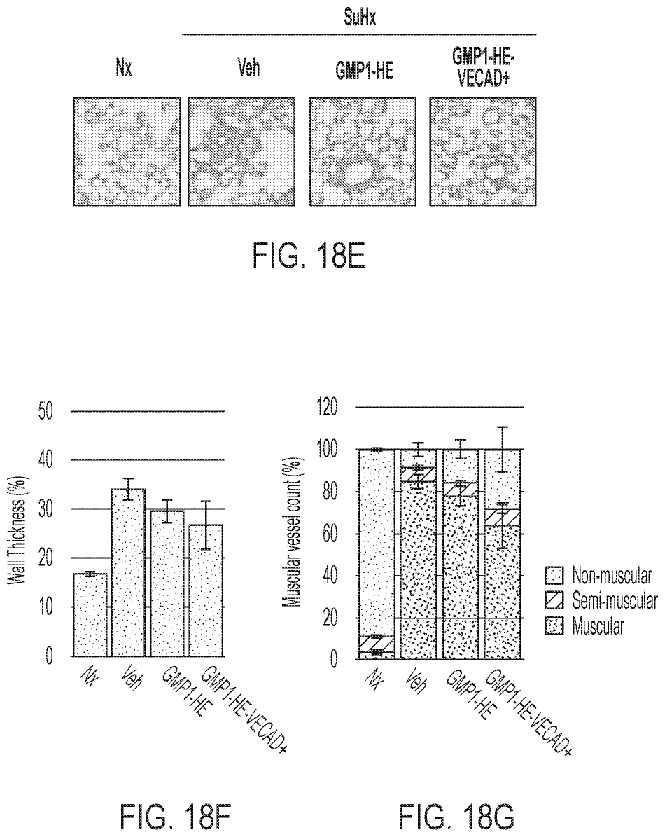

The single cell profiling of HE and the similarity between the VECAD+HE fraction and HUVEC described above suggested that perhaps this subpopulation could be an active component conferring HE its therapeutic effects in PAH. To test this, another study using the Sugen/hypoxia model was performed using 2.5 million “bulk” or unsorted HE cells and 2.5 million VECAD+HE cells purified from the “bulk” HE cells by magnetic sorting for VECAD+ cells (FIG. 17). The fraction sorted for VECAD+ cells showed that the majority also express CD31 (FIG. 17). Compared to vehicle-treated animals, VECAD+HE improved clinical measurements: mPAP (FIG. 18A), RVSP (FIG. 18B), RV remodeling (FIG. 18C) and cardiac output (FIG. 18D). The lung vasculature was also maintained compared to vehicle-treated, with fewer plexiform lesions (FIG. 18E), reduced wall thickness (FIG. 18F), and reduced vessel muscularization (FIG. 18G). Similar results were obtained by delivery of bona fide mature endothelial cells, HUVEC.

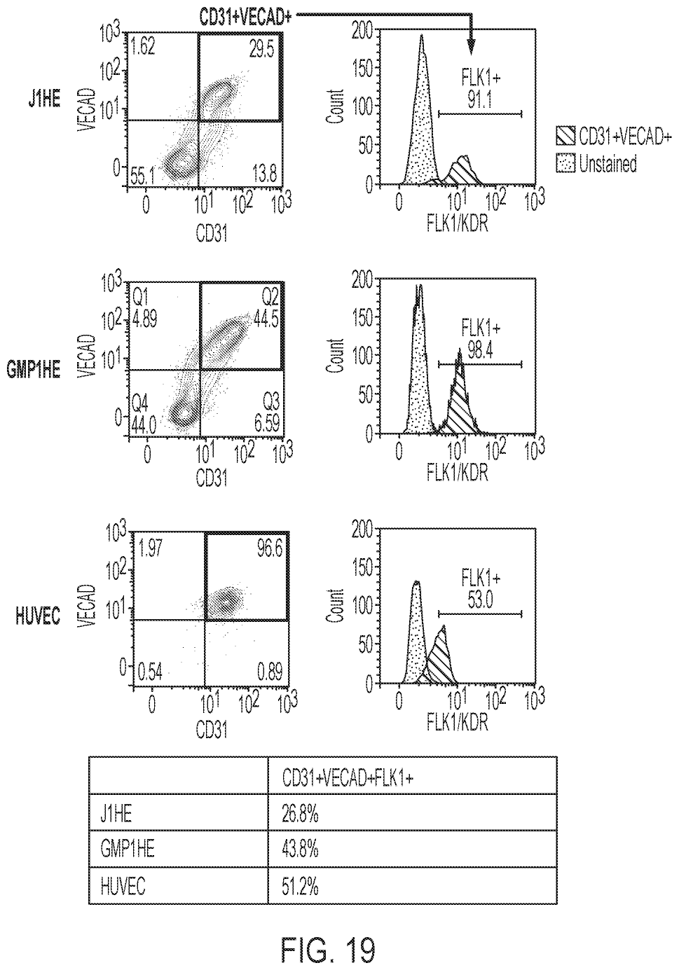

When the VECAD+/CD31+ populations in J1-HEs and GMP1-HEs were analyzed for FLK1/KDR expression, the HEs were shown to comprise a population that was CD31+/VECAD+/FLK1+(FIG. 19).

### EQUIVALENTS

Those skilled in the art will recognize, or be able to ascertain using no more than routine experimentation, many equivalents to the specific embodiments of the invention described herein. such equivalents are intended to be encompassed by the following claims. The contents of all references, patents and published patent applications cited throughout this application are incorporated herein by reference.

1. A method of treating a vascular disease in a subject suffering from, or suspected of suffering from, a vascular disease, comprising administering to the subject a composition comprising hemogenic endothelial cells (HEs) obtained by in vitro differentiation of pluripotent stem cells.

2. The method of claim 1, wherein the vascular disease is selected from the group consisting of coronary artery diseases (e.g., arteriosclerosis, atherosclerosis, and other diseases or injuries of the arteries, arterioles and capillaries or related complaint), myocardial infarction, (e.g. acute myocardial infarction), organizing myocardial infarct, ischemic heart disease, arrhythmia, left ventricular dilatation, emboli, heart failure, congestive heart failure, subendocardial fibrosis, left or right ventricular hypertrophy, myocarditis, chronic coronary ischemia, dilated cardiomyopathy, restenosis, arrhythmia, angina, hypertension (eg. pulmonary hypertension, glomerular hypertension, portal hypertension), myocardial hypertrophy, peripheral arterial disease including critical limb ischemia, cerebrovascular disease, renal artery stenosis, aortic aneurysm, pulmonary heart disease, cardiac dysrhythmias, inflammatory heart disease, congenital heart disease, rheumatic heart disease, diabetic vascular diseases, endothelial lung injury diseases (e.g., acute lung injury (ALI), and acute respiratory distress syndrome (ARDS)).

3. The method of claim 1, wherein the vascular disease is pulmonary hypertension or pulmonary arterial hypertension.

4. (canceled)

5. The method of claim 1, wherein the mean pulmonary (artery) pressure is reduced in the subject.

6. A method of increasing blood flow in pulmonary arteries in a subject suffering from, or suspected of suffering from, a vascular disease, comprising administering to the subject a composition comprising HEs obtained by in vitro differentiation of pluripotent stem cells.

7. The method of claim 6, wherein the subject has pulmonary hypertension or pulmonary arterial hypertension.

8. (canceled)

9. A method of reducing blood pressure in a subject suffering from, or suspected of suffering from, a vascular disease, comprising administering to the subject a composition comprising HEs obtained by in vitro differentiation of pluripotent stem cells.

10. The method of claim 9, wherein the subject has pulmonary hypertension or pulmonary arterial hypertension.

11. (canceled)

12. The method of claim 9, wherein the blood pressure is diastolic pressure, systolic pressure, and/or mean pulmonary (artery) pressure.

13. (canceled)

14. (canceled)

15. The method of claim 9, wherein the blood pressure is reduced by at least 20% in the subject.

16. The method of claim 1, wherein (a) the HEs are positive for at least one microRNA (miRNA) selected from the group consisting of miRNA-126, miRNA-24, miRNA-196-b, miRNA-214, miRNA-199a-3p, miRNA-335, hsa-miR-11399, hsa-miR-196b-3p, hsa-miR-5690, and hsa-miR-7151-3p;
(b) the HEs are positive for (i) miRNA-214, miRNA-199a-3p, and miRNA-335 and/or (ii) hsa-miR-11399, hsa-miR-196b-3p, hsa-miR-5690, and hsa-miR-7151-3p;
(c) the HEs are positive for (i) miRNA-126, miRNA-24, miRNA-196-b, miRNA-214, miRNA-199a-3p, and miRNA-335 and/or (ii) hsa-miR-11399, hsa-miR-196b-3p, hsa-miR-5690, and hsa-miR-7151-3p;
(d) the HEs are positive for miRNA-214;
(e) the HEs are negative for at least one miRNA selected from the group consisting of miRNA-367, miRNA-302a, miRNA-302b, miRNA-302c, miRNA-223, and miRNA-142-3p;
(f) the HEs are negative for miRNA-223, and miRNA-142-3p;
(g) the HEs are negative for miRNA-367, miRNA-302a, miRNA-302b, miRNA-302c, miRNA-223, and miRNA-142-3p;
(h) the HEs express at least one cell surface marker selected from the group consisting of CD31/PECAM1, CD309/KDR, CD144, CD34, CXCR4, CD146, Tie2, CD140b, CD90, CD271, and CD105;
(i) the HEs express CD146, CXCR4, CD309/KDR, CD90, and CD271;
(j) the HEs express CD146;
(k) the HEs express CD144 (VECAD);
(l) the HEs express at least one cell marker selected from the group consisting of CD31, CD309/KDR (FLK-1), PLVAP, GJA4, ESAM, EGFL7, KDR/VEGFR2, and ESAM;
(m) the HEs further express at least one cell marker selected from the group consisting of SOX9, PDGFRA, and EGFRA;
(n) the HEs further express at least one cell marker selected from the group consisting of KDR/VEGFR2, NOTCH4, collagen I, and collagen IV;
(o) the HEs express CD31/PECAM1, CD309/KDR, CD144, CD34, and CD105;
(p) the HEs exhibit limited or no detection of at least one cell surface marker selected from the group consisting of CD34, CXCR7, CD43 and CD45;
(q) the HEs exhibit limited or no detection of CXCR7, CD43, and CD45;
(r) the HEs exhibit limited or no detection of CD43 and CD45;
(s) the HEs are CD43(−), CD45(−), and CD146 (+);
(t) the HEs express (i) CD144 (VECAD) and (ii) CD31 and/or CD309/KDR (FLK-1); and/or
(u) the HEs express at least one gene listed in Table 22 and Table 23.

17-34. (canceled)

35. The method of claim 1, wherein the pluripotent stem cells are embryonic stem cells or pluripotent stem cells.

36. (canceled)

37. The method of claim 1, wherein the HEs are obtained (i) by culturing the pluripotent stem cells under adherent conditions in a differentiation medium in the absence of methylcellulose; and/or (ii) by in vitro differentiation of pluripotent stem cells without embryoid body formation.

38. (canceled)

39. The method of claim 1, wherein the subject is a human.

40. (canceled)

41. (canceled)

42. A composition comprising HEs obtained by in vitro differentiation of pluripotent stem cells, wherein the HEs are CD43(−), CD45(−), and CD146 (+).

43. A composition comprising HEs obtained by in vitro differentiation of pluripotent stem cells, wherein the HEs are positive for at least one microRNA (miRNA) selected from the group consisting of miRNA-126, mi-RNA-24, miRNA-196-b, miRNA-214, miRNA-199a-3p, miRNA-335, hsa-miR-11399, hsa-miR-196b-3p, hsa-miR-5690, and hsa-miR-7151-3p.

44. The composition of claim 43, wherein (a) the HEs are positive for (i) miRNA-214, miRNA-199a-3p, and miRNA-335 and/or (ii) hsa-miR-11399, hsa-miR-196b-3p, hsa-miR-5690, and hsa-miR-7151-3p;
(b) the HEs are positive for (i) miRNA-126, mi-RNA-24, miRNA-196-b, miRNA-214, miRNA-199a-3p, and miRNA-335 and/or (ii) hsa-miR-11399, hsa-miR-196b-3p, hsa-miR-5690, and hsa-miR-7151-3p;
(c) the HEs are positive for miRNA-214;
(d) the HEs are negative for at least one miRNA selected from the group consisting of miRNA-367, miRNA-302a, miRNA-302b, miRNA-302c, miRNA-223, and miRNA-142-3p;
(e) the HEs are negative for miRNA-223, and miRNA-142-3p;
(f) the HEs are negative for miRNA-367, miRNA-302a, miRNA-302b, miRNA-302c, miRNA-223, and miRNA-142-3p; and/or
(g) the HEs are CD43(−), CD45(−), and CD146 (+).

45-50. (canceled)

51. A composition comprising HEs obtained by in vitro differentiation of pluripotent stem cells, wherein the HEs express CD144 (VECAD).

52. The composition of claim 51, wherein (a) the HEs further express at least one cell marker selected from the group consisting of CD31, CD309/KDR (FLK-1), PLVAP, GJA4, ESAM, EGFL7, KDR/VEGFR2, and ESAM;
(b) the HEs further express at least one cell marker selected from the group consisting of SOX9, PDGFRA, and EGFRA;
(c) the HEs further express at least one cell marker selected from the group consisting of KDR/VEGFR2, NOTCH4, collagen I, and collagen IV;
(d) the composition substantially lacks CD144 (VECAD)-negative HE cells;
(e) the HEs express (i) CD144 (VECAD) and (ii) CD31 and/or CD309/KDR (FLK-1); and/or
(f) the HEs express at least one gene listed in Table 22 and Table 23.

53-55. (canceled)

56. A pharmaceutical composition comprising HEs obtained by in vitro differentiation of pluripotent stem cells and a pharmaceutically acceptable carrier, wherein the HEs are CD43(−), CD45(−), and CD146 (+).

57. A pharmaceutical composition comprising HEs obtained by in vitro differentiation of pluripotent stem cells and a pharmaceutically acceptable carrier, wherein the HEs are positive for at least one microRNA (miRNA) selected from the group consisting of miRNA-126, mi-RNA-24, miRNA-196-b, miRNA-214, miRNA-199a-3p, miRNA-335, hsa-miR-11399, hsa-miR-196b-3p, hsa-miR-5690, and hsa-miR-7151-3p.

58. The pharmaceutical composition of claim 57, wherein (a) the HEs are positive for (i) miRNA-214, miRNA-199a-3p, and miRNA-335 and/or (ii) hsa-miR-11399, hsa-miR-196b-3p, hsa-miR-5690, and hsa-miR-7151-3p;
(b) the HEs are positive for (i) miRNA-126, mi-RNA-24, miRNA-196-b, miRNA-214, miRNA-199a-3p, and miRNA-335 and/or (ii) hsa-miR-11399, hsa-miR-196b-3p, hsa-miR-5690, and hsa-miR-7151-3p;
(c) the HEs are positive for miRNA-214;
(d) the HEs are negative for at least one miRNA selected from the group consisting of miRNA-367, miRNA-302a, miRNA-302b, miRNA-302c, miRNA-223, and miRNA-142-3p;
(e) the HEs are negative for miRNA-223, and miRNA-142-3p;
(f) the HEs are negative for miRNA-367, miRNA-302a, miRNA-302b, miRNA-302c, miRNA-223, and miRNA-142-3p; and/or
(g) the HEs are CD43(−), CD45(−), and CD146 (+).

59-64. (canceled)

65. A pharmaceutical composition comprising HEs obtained by in vitro differentiation of pluripotent stem cells and a pharmaceutically acceptable carrier, wherein the HEs express CD144 (VECAD), CD31, and CD309/KDR (FLK-1).

66. The pharmaceutical composition of claim 65, wherein (a) the HEs further express at least one cell marker selected from the group consisting of CD31, CD309/KDR (FLK-1), PLVAP, GJA4, ESAM, EGFL7, KDR/VEGFR2, and ESAM;
(b) the HEs further express at least one cell marker selected from the group consisting of SOX9, PDGFRA, and EGFRA;
(c) the HEs further express at least one cell marker selected from the group consisting of KDR/VEGFR2, NOTCH4, collagen I, and collagen IV;
(d) the composition substantially lacks CD144 (VECAD)-negative HE cells;
(e) the HEs express (i) CD144 (VECAD) and (ii) CD31 and/or CD309/KDR (FLK-1); and/or
(f) the HEs express at least one gene listed in Table 22 and Table 23.

67-75. (canceled)
